(19) United States
(12) Patent Application Publication
(10) Pub. No.: US 2025/0109435 A1
(43) Pub. Date: Apr. 3, 2025

(54) METHODS FOR MULTIPLEXED PROTEIN DETECTION

(71) Applicant: GUARDANT HEALTH, INC., Palo Alto, CA (US)

(72) Inventors: James GHADIALI, San Diego, CA (US); Andrew KENNEDY, San Diego, CA (US)

(21) Appl. No.: 18/900,488

(22) Filed: Sep. 27, 2024

Related U.S. Application Data
(60) Provisional application No. 63/586,062, filed on Sep. 28, 2023.

(51) Int. Cl. C12Q 1/6869; C12Q 1/6804; G01N 33/53; G01N 33/543; G01N 33/574

(52) U.S. Cl. CPC C12Q 1/6869; C12Q 1/6804; G01N 33/5308; G01N 33/54326; G01N 33/57488; G01N 2458/10

(57) ABSTRACT

Disclosed are methods for multiplexed protein detection, including, for example, a multiplex immunoassay capable of eliminating or reducing false positives in proximity probe-based assays. The disclosure also provides methods for reducing noise due to probe and oligo cross-reactivity. Additional methods to integrate protein readouts with mulitiomics datasets and related systems and computer-readable media are also provided.

### CROSS-REFERENCE TO RELATED APPLICATIONS

This application claims the benefit of U.S. Provisional Patent Application No. 63/586,062, filed on Sep. 28, 2023, and is incorporated by reference herein in its entirety.

### BACKGROUND

Massive parallel sequencing has proven revolutionary in the study of common complex diseases, identifying a vast number of genomic markers. However, genomic, and transcriptomic data may not always reflect the corresponding protein changes. Additionally, these data ignore posttranslational modifications (PTMs), including phosphorylation and ubiquitination.

Hence, there is a need to integrate highly sensitive proteomic readouts with molecular phenotypes, such as epigenomic data, transcriptome profiling, or metabolomic data. This approach can potentially discover new pathways that control malignant transformation, disease onset, progression, reoccurrence, and therapeutic outcomes.

Additionally, improvements are needed to reduce or prevent false positives. In current proteomic assays for example, target binding, extension, and subsequent amplification reactions occur in solution. This one-step, in solution, assay approach renders the immunoassay and molecular biology steps sensitive to matrix components (e.g., due to variability in sample composition, such as plasma). In addition, as both detection antibodies are present contemporaneously, there is potential for antibody cross-hybridization in the absence of analyte, increasing the risk of false positive signals that confound analysis or contribute to the overall cost. Thus, there is a need for methods to eliminate or reduce false positives in proximity probe-based assays.

### BRIEF SUMMARY

The present disclosure addresses deficiencies of the prior methods by providing a multiplex immunoassay capable of eliminating/reducing false positives in proximity probe-based assays. The present disclosure provides methods for significantly reducing noise due to protein-antibody and oligo-binding cross-reactivity.

Disclosed are methods comprising contacting a sample comprising one or more target analytes with at least one first probe, wherein the first probe comprises a ligand, an analyte binding domain and a polynucleotide, wherein the first probe specifically binds to one of the target analytes in the sample, thereby generating a first complex comprising the target analyte bound to the analyte binding domain of the first probe; contacting the first complex with a capture molecule, wherein the capture molecule binds to the ligand of the first probe of the first complex, thereby generating a captured first complex; contacting the captured first complex with at least one second probe, wherein the second probe comprises an analyte binding domain specific for the target analyte in the captured first complex and a polynucleotide, wherein the polynucleotide of the second probe comprises a sequence complementary to a portion of the polynucleotide of the first probe, wherein the second probe specifically binds to the target analyte in the captured first complex, thereby generating at least one second complex; incubating the second complex under conditions to allow for hybridization of the polynucleotide of the first probe to the polynucleotide of the second probe; and extending the hybridized polynucleotides of the first and second probes to generate complementary strands of the polynucleotides of the first and second probes.

Disclosed are methods of contacting a sample comprising one or more target analytes with at least one first probe, wherein the first probe comprises a capture molecule, a ligand, an analyte binding domain and a polynucleotide, wherein the ligand is bound to the capture molecule, wherein the first probe specifically binds to one of the target analytes in the sample, thereby generating a captured first complex comprising the target analyte bound to the analyte binding domain of the first probe; contacting the captured first complex with at least one second probe, wherein the second probe comprises an analyte binding domain specific for the target analyte in the captured first complex and a polynucleotide, wherein the polynucleotide of the second probe comprises a sequence complementary to a portion of the polynucleotide of the first probe, wherein the second probe specifically binds to the target analyte in the captured first complex, thereby generating at least one second complex; incubating the second complex under conditions to allow for hybridization of the polynucleotide of the first probe to the polynucleotide of the second probe; and extending the hybridized polynucleotides of the first and second probes to generate complementary strands of the polynucleotides of the first and second probes.

Disclosed are methods of contacting a sample comprising one or more target analytes with at least one first probe, wherein the first probe comprises a capture molecule, an analyte binding domain and a polynucleotide, wherein the first probe specifically binds to one of the target analytes in the sample, thereby generating a captured first complex comprising the target analyte bound to the analyte binding domain of the first probe; contacting the captured first complex with at least one second probe, wherein the second probe comprises an analyte binding domain specific for the target analyte in the captured first complex and a polynucleotide, wherein the polynucleotide of the second probe comprises a sequence complementary to a portion of the polynucleotide of the first probe, wherein the second probe specifically binds to the target analyte in the captured first complex, thereby generating at least one second complex; incubating the second complex under conditions to allow for hybridization of the polynucleotide of the first probe to the polynucleotide of the second probe; and extending the hybridized polynucleotides of the first and second probes to generate complementary strands of the polynucleotides of the first and second probes.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1A shows sources of cross-reactivity noise and false positives in standard probe-based immunoassays.

FIG. 1B shows additional undesired cross-reactivities in standard probe-based immunoassays.

FIG. 1C shows sources of cross-reactivity noise and false positives in standard bead-based immunoassays.

FIG. 2 shows an immunoassay workflow using an immobilized antibody-oligo probe to remove background signal. The workflow allows the introduction of a wash step to purify antibody-target protein complex while retaining or improving the specificity readout of standard immunoassays.

FIG. 3 shows an immunoassay workflow using an immobilized antibody-oligo probe to remove background signal. The illustrated workflow shows a capture antibody is pre-complexed with a solid support.

FIG. 4 shows an immunoassay workflow using multiplexed immuno-PCR with solid support and qPCR/NGS readout with a direct complex between capture Ab complex and solid support and only a polynucleotide on a single probe.

FIG. 5 shows an immunoassay workflow using multiplexed immuno-PCR with solid support and qPCR/NGS readout with a capture Ab complexed to solid support through universal secondary Ab-bead complex and only a polynucleotide on a single probe.

FIG. 6 shows an immunoassay workflow using a tertiary-blocking oligonucleotide that reversibly associates with the antibody-linked oligonucleotide. This diagram illustrates a workflow that prevents direct hybridization of the two antibody-linked oligonucleotides.

FIGS. 7A-7C show additional methods to increase the likelihood that capture oligo will be blocked during primary immuno-incubation. (FIG. 7A) A cis-blocking sequence is added to the blocking oligo, (FIG. 7B) universal-secondary antibody bead capture complex, or (FIG. 7C) the capture oligo is conjugated directly capture particle.

FIG. 8 shows Ab-oligo design specification and in-process UDG step addition can reduce false positives

FIGS. 9A and 9B show methods to (FIG. 9A) detect signal from protein isoforms and post-translational modifications and to (FIG. 9B) detect detection antibody cross-reactivity to improve sensitivity.

FIG. 10 shows an exemplary qPCR signal from IL-6 titration in solid-phase immunoassay including blocking oligo protection.

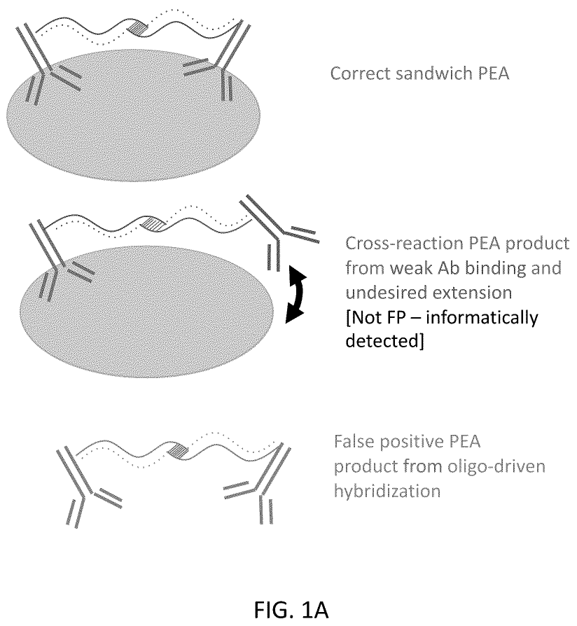

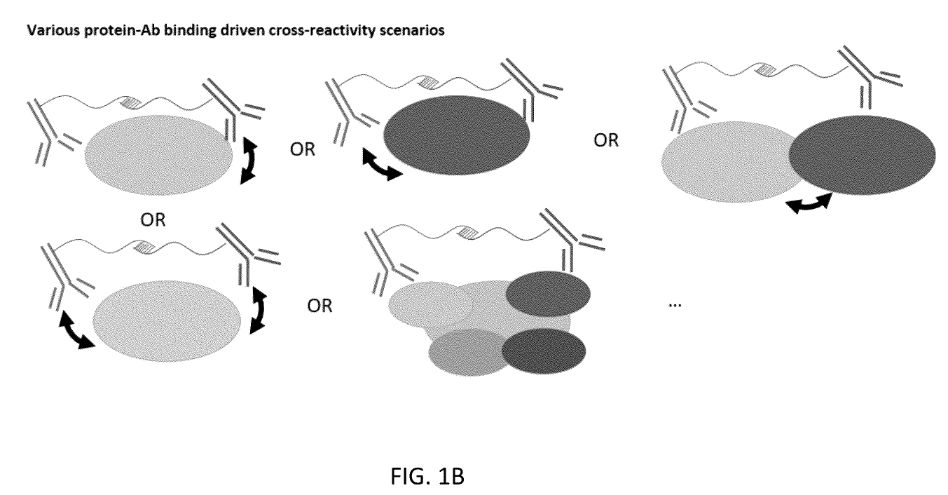

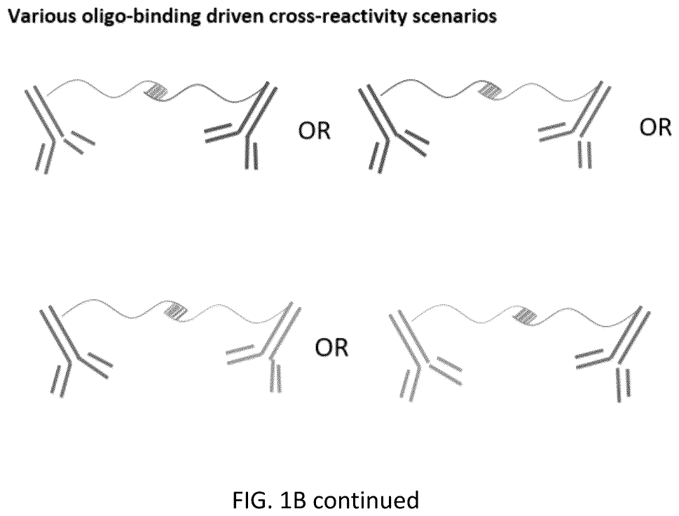

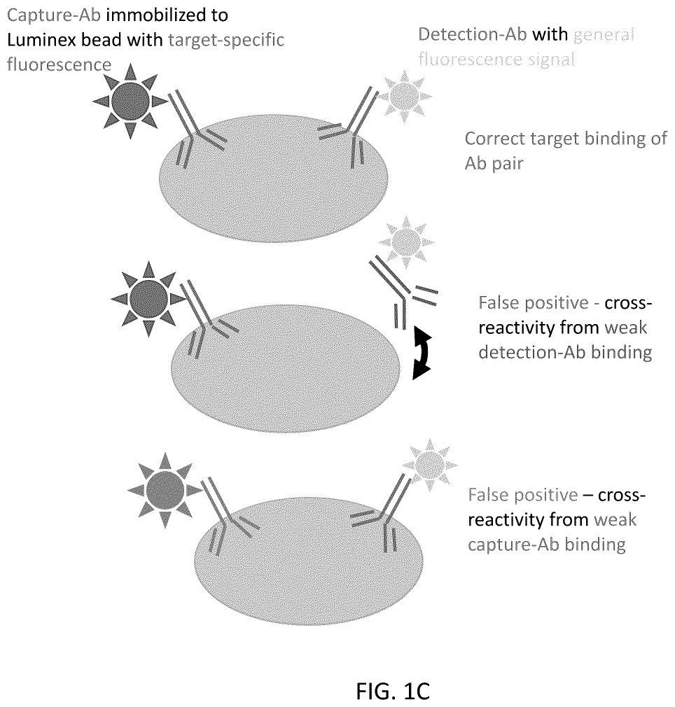

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and Care disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Headings are provided for convenience only and are not to be construed to limit the invention in any manner. Embodiments illustrated under any heading or in any portion of the disclosure may be combined with embodiments illustrated under the same or any other heading or other portion of the disclosure.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a first probe” can include a plurality of such first probes, reference to “the target analyte” is a reference to one or more target analytes and equivalents thereof known to those skilled in the art, and so forth.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Methods

By eliminating or reducing false positives in proximity extension assays, the techniques disclosed herein may reduce the time and effort needed to analyze samples. In the process, the analysis technique may increase confidence in assays, such as proteomic assays. Moreover, the analysis techniques may facilitate early detection of complex diseases (such as cancer, cardiovascular, asthma, diabetes, epilepsy, hypertension, manic depression, Alzheimer's, and schizophrenia), and may provide improved diagnosis, tracking of disease progression and treatment. Furthermore, the analysis techniques may enable further understanding of a variety of types of cancer, and may facilitate the development of new treatments or therapeutic interventions. Consequently, the analysis techniques may reduce unnecessary or untimely therapeutic interventions, patient suffering, and patient mortality.

The disclosed methods can detect the presence of molecules such as proteins and/or nucleic acids in a sample. In some aspects, the presence of these molecules can be used to diagnose or prognose disease.

The present methods can be used to diagnose the presence of conditions, particularly cancer, in a subject, to characterize conditions (e.g., staging cancer or determining heterogeneity of a cancer), monitor response to treatment of a condition, effect prognosis risk of developing a condition or subsequent course of a condition. The present disclosure can also be useful in determining the efficacy of a particular treatment option. Successful treatment options may increase the amount of copy number variation or rare mutations detected in subject's blood if the treatment is successful as more cancers may die and shed DNA. In other aspects, this may not occur. In another example, perhaps certain treatment options may be correlated with genetic and/or proteomic profiles of cancers over time. This correlation may be useful in selecting a therapy. Additionally, if a cancer is observed to be in remission after treatment, the present methods can be used to monitor residual disease or recurrence of disease.

The types and number of cancers that may be detected by the methods disclosed herein may include blood cancers, breast cancers, brain and central nervous system (cns) cancers, lung cancers, skin cancers, nose cancers, prostate cancers, colorectal cancers, throat cancers, liver cancers, bone cancers, lymphomas, pancreatic cancers, skin cancers, bowel cancers, rectal cancers, thyroid cancers, bladder cancers, ovarian cancers, cervical cancers, uterine (endometrial) cancers, kidney cancers, lymphoma, multiple myeloma, esophageal cancer, mouth cancers, stomach cancers, soft tissue sarcoma, mesothelioma, head and neck cancers, testicular cancer, eye cancer, gastrointestinal stromal tumor (gist), adrenal gland cancer, melanoma, or glioblastoma), and/or cancers exhibiting cancer markers, such as: Her2, CA15-3, CA19-9, CA-125, CEA, AFP, PSA, HCG, hormone receptor and NMP-22, solid state tumors, heterogeneous tumors, homogenous tumors and the like. The disclosed methods can be used to determine or detect the type and/or stage of cancer from genetic variations including mutations, rare mutations, indels, copy number variations, transversions, translocations, inversion, deletions, ancuploidy, partial ancuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine.

As used herein, “common complex diseases” can include various diseases including cancer, cardiovascular disease, and neurological diseases.

Neurological complex diseases can include Alzheimer's disease, epilepsy, schizophrenia, autism spectrum disorders, bipolar disorder, Tourette syndrome, and attention-deficit/hyperactivity disorder (ADHD).

Cardiovascular disease (CVD) can include coronary artery disease, heart failure, stroke, atrial fibrillation, hypertension, peripheral artery disease, arrhythmias, valvular heart disease, cardiomyopathies, and congenital heart diseases.

The present disclosure is also described and demonstrated by way of the following embodiments. However, the use of these and other embodiments anywhere in the specification is illustrative only and in no way limits the scope and meaning of a claimed invention or of any exemplified term. Likewise, a claimed invention is not limited to any preferred embodiment described herein. Indeed, many modifications and variations of a claimed invention may be apparent to those skilled in the art upon reading this specification, and such variations can be made without departing from that claimed invention in spirit or in scope. A claimed invention is therefore to be limited only by the terms of the appended claims along with the full scope of equivalents to which those claims are entitled.

In some aspects, the disclosed methods use one or more the samples described herein and wherein the sample comprises one or more of the target analytes described herein.

a. Sample

In some aspects, a sample can be any biological sample isolated from a subject. A sample can be a bodily sample. Samples can include body tissues, such as known or suspected solid tumors, serum, plasma, blood, cerebrospinal fluid (csf), amniotic fluid, synovial fluid, lymph, interstitial fluid, intracellular fluid (icf), saliva, urine, bile, feces, tears, sputum, sweat, mucus, gastric juice, semen, vaginal fluid, breast milk, aqueous humor, peritoneal fluid, pleural fluid, pericardial fluid, endolymph, perilymph, vitreous humor, pancreatic juice, bronchoalveolar lavage fluid (bal), synovial fluid, meconium, follicular fluid, ejaculate, amniotic fluid, ascitic fluid, gingival crevicular fluid (gcf). Examples of solid tumor tissues include, carcinoma, sarcoma, lymphoma, melanoma, glioma, adenoma, fibroma, lipoma, ostcoma, chondroma, teratoma, neuroblastoma, hepatoma, nephroblastoma, rhabdomyosarcoma, medulloblastoma, ependymoma, meningioma, thymoma, neurofibroma, pheochromocytoma, paraganglioma, schwannoma, desmoid tumor, angiosarcoma, leiomyosarcoma, mesothelioma, chordoma, hemangioma, retinoblastoma, seminoma, germ cell tumor, pancreatic neuroendocrine tumor, small cell lung cancer, non-small cell lung cancer, hepatocellular carcinoma, renal cell carcinoma, colorectal adenocarcinoma, ovarian carcinoma, cervical carcinoma, uterine carcinoma, breast carcinoma, prostate carcinoma, testicular carcinoma, thyroid carcinoma, esophageal carcinoma, stomach carcinoma, bladder carcinoma, head and neck squamous cell carcinoma. Samples are preferably body fluids, particularly blood and fractions thereof, and urine. In some aspects, a sample can be in the form originally isolated from a subject or can have been subjected to further processing to remove or add components, such as cells, or enrich for one component relative to another. Thus, a preferred body fluid for analysis is plasma or serum containing cell-free nucleic acids. In some aspects, a sample can be isolated or obtained from a subject and transported to a site of sample analysis. In some aspects, the sample can be preserved and shipped at a desirable temperature, e.g., room temperature, 4° C., −20° C., and/or −80° C. In some aspects, a sample can be isolated or obtained from a subject at the site of the sample analysis. In some aspects, the subject can be a human, a mammal, an animal, a companion animal, a service animal, or a pet. The subject may have a cancer. The subject may not have cancer or a detectable cancer symptom. In some aspects, the subject may have been treated with one or more cancer therapies, e.g., any one or more of chemotherapies, antibodies, vaccines or biologics. In some aspects, the subject may be in remission. In some aspects, the subject may or may not be diagnosed of being susceptible to cancer or any cancer-associated genetic mutations/disorders.

In some aspects, the sample can be plasma. The volume of plasma can depend on the desired read depth for sequenced regions. Exemplary volumes are 0.4-40 mL, 5-20 mL, 10-20 mL. For examples, the volume can be 0.5 mL, 1 mL, 5 mL 10 mL, 20 mL, 30 mL, or 40 mL. A volume of sampled plasma may be 5 to 20 mL.

In some aspects, the term sample can be used to refer to extractions of molecules from within a biological sample. For example, in some aspects, the sample can be proteins or nucleic acids present in a biological sample obtained from a subject. Thus, in some aspects, disclosed are methods of extracting a sample from a biological sample. Protein extraction methods encompass various approaches, from mechanical homogenization, chemical lysis, and enzymatic digestion to fractionation techniques, precipitation methods, and immunoaffinity purification. Mechanical homogenization, such as bead beating or ultrasonic homogenization, physically disrupts cells and tissues. Chemical lysis involves alkaline, acidic, or organic solvent-based methods. Enzymatic digestion, like trypsin or proteinase K digestion, breaks down proteins into smaller fragments. Precipitation methods, such as salting out or TCA precipitation, separate proteins from other components. Detergent-based approaches like SDS extraction and fractionation techniques like gel filtration chromatography are also common. Additionally, commercially available protein extraction kits include: for total protein extraction: Thermo Fisher Scientific's NE-PER™ Nuclear and Cytoplasmic Extraction Reagents, Bio-Rad's Total Protein Extraction Kit, and Abcam's Total Protein Extraction Kit. To isolate specific cellular fractions: QIAGEN's Qproteome Mammalian Protein Prep Kit for cell and tissue lysis, to isolate subcellular fractionation kits like Abcam's Mitochondria Isolation Kit. Immunoprecipitation (IP) kits, solid-phase extraction kits, and enzyme-specific digestion kits, are also known in the art an manufactured by Thermo Fisher Scientific, Promega, and Roche.

b. Target Analyte

In some aspects, the sample comprises one or more target analytes. In general, the disclosed systems, apparatus, methods, and compositions can be used to analyze any number of analytes, including both nucleic acid analytes and non-nucleic acid analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate. Methods for performing multiplexed assays to analyze two or more different analytes re disclosed herein.

Analytes can include nucleic acid analytes, and non-nucleic acid analytes. This disclosure provides for detecting genetic variations in biological samples from a subject. Biological samples may include polynucleotides from cancer cells. Polynucleotides may be DNA (e.g., genomic DNA, cDNA), RNA (e.g., mRNA, small RNAs), or any combination thereof. Biological samples may include tumor tissue, e.g., from a biopsy. In some cases, biological samples may include blood or saliva. In particular cases, biological samples may comprise cell free DNA (“cfDNA”) or circulating tumor DNA (“ctDNA”). Cell free DNA can be present in, e.g., blood.

Exemplary amounts of cell-free nucleic acids in a sample before amplification range from about 1 fg to about 1 μg, e.g., 1 μg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of cell-free nucleic acid molecules. The amount can be at least 1 fg, at least 10 fg, at least 100 fg, at least 1 pg, at least 10 pg, at least 100 pg, at least 1 ng, at least 10 ng, at least 100 ng, at least 150 ng, or at least 200 ng of cell-free nucleic acid molecules. The amount can be up to 1 femtogram (fg), 10 fg, 100 fg, 1 picogram (pg), 10 pg, 100 pg, 1 ng, 10 ng, 100 ng, 150 ng, or 200 ng of cell-free nucleic acid molecules. The method can comprise obtaining 1 femtogram (fg) to 200 ng.

Cell-free nucleic acids are nucleic acids not contained within or otherwise bound to a cell or in other words nucleic acids remaining in a sample after removing intact cells. Cell-free nucleic acids include DNA, RNA, and hybrids thereof, including genomic DNA, mitochondrial DNA, siRNA, miRNA, circulating RNA (cRNA), IRNA, rRNA, small nucleolar RNA (snoRNA), Piwi-interacting RNA (piRNA), long non-coding RNA (long ncRNA), or fragments of any of these. Cell-free nucleic acids can be double-stranded, single-stranded, or a hybrid thereof. A cell-free nucleic acid can be released into bodily fluid through secretion or cell death processes, e.g., cellular necrosis and apoptosis. Some cell-free nucleic acids are released into bodily fluid from cancer cells e.g., circulating tumor DNA, (ctDNA). Others are released from healthy cells. In some embodiments, cfDNA is cell-free fetal DNA (cffDNA) In some embodiments, cell free nucleic acids are produced by tumor cells. In some embodiments, cell free nucleic acids are produced by a mixture of tumor cells and non-tumor cells.

Cell-free nucleic acids have an exemplary size distribution of about 100-500 nucleotides, with molecules of 110 to about 230 nucleotides representing about 90% of molecules, with a mode of about 168 nucleotides and a second minor peak in a range between 240 to 440 nucleotides. Cell-free nucleic acids can be isolated from bodily fluids through a fractionation or partitioning step in which cell-free nucleic acids, as found in solution, are separated from intact cells and other non-soluble components of the bodily fluid. Partitioning may include techniques such as centrifugation or filtration. Alternatively, cells in bodily fluids can be lysed and cell-free and cellular nucleic acids processed together. Generally, after addition of buffers and wash steps, nucleic acids can be precipitated with an alcohol. Further clean up steps may be used such as silica based columns to remove contaminants or salts. Non-specific bulk carrier nucleic acids, such as Cot-1 DNA, DNA or protein for bisulfite sequencing, hybridization, and/or ligation, may be added throughout the reaction to optimize certain aspects of the procedure such as yield.

After such processing, samples can include various forms of nucleic acid including double stranded DNA, single stranded DNA and single stranded RNA. In some embodiments, single stranded DNA and RNA can be converted to double stranded forms so they are included in subsequent processing and analysis steps.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquity lati on variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. This further includes receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

In some aspects, a sample can further comprise one or more non-target analytes. Non-target analytes can be removed during the disclosed methods so as to not interfere with results of the methods.

1. Bind First Probe to Target Analyte then Use a Capture Molecule

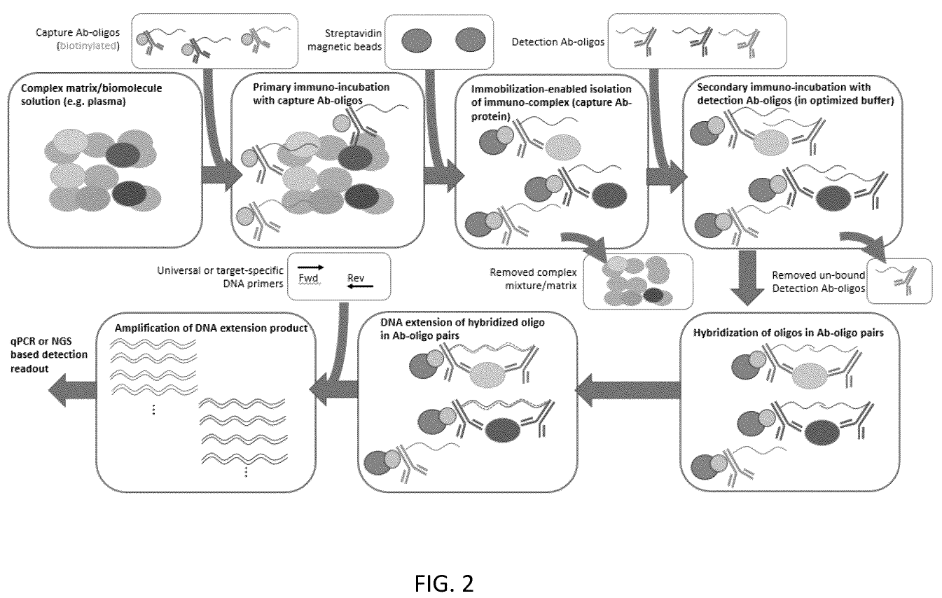

An example of the disclosed methods is illustrated in FIG. 2. A broader description of the method is as follows.

Disclosed are methods comprising contacting a sample comprising one or more target analytes with at least one first probe, wherein the first probe comprises a ligand, an analyte binding domain and a polynucleotide, wherein the first probe specifically binds to one of the target analytes in the sample, thereby generating a first complex comprising the target analyte bound to the analyte binding domain of the first probe; contacting the first complex with a capture molecule, wherein the capture molecule binds to the ligand of the first probe of the first complex, thereby generating a captured first complex; contacting the captured first complex with at least one second probe, wherein the second probe comprises an analyte binding domain specific for the target analyte in the captured first complex and a polynucleotide, wherein the polynucleotide of the second probe comprises a sequence complementary to a portion of the polynucleotide of the first probe, wherein the second probe specifically binds to the target analyte in the captured first complex, thereby generating at least one second complex; incubating the second complex under conditions to allow for hybridization of the polynucleotide of the first probe to the polynucleotide of the second probe; and extending the hybridized polynucleotides of the first and second probes to generate complementary strands of the polynucleotides of the first and second probes.

In some aspects, incubating the second complex under conditions to allow for hybridization of the polynucleotide of the first probe to the polynucleotide of the second probe at the complementary sequences of each. In some aspects, the result is a duplex of the polynucleotide of the first probe to the polynucleotide of the second probe. In some aspects, the 3′ end of the polynucleotide of the second probe hybridizes to the 3′ end of the polynucleotide of the first probe. Thus, in some aspects, the sequence of the second probe that is complementary to a portion of the polynucleotide of the first probe is present at the 3′ end of the polynucleotide of the second probe.

In some aspects, extending the hybridized polynucleotides of the first and second probes to generate complementary strands of the polynucleotides of the first and second probes comprises incubating the at least one second complex having hybridized polynucleotides with a solution comprising at least an extending polymerase and nucleotides allowing extension of the hybridized polynucleotides. In some aspects, the extension can be performed under the same or similar conditions as the extension step of known proximity extension assays. In some aspects, one or both of the polynucleotides of the first and second probes are extended. For example, if the polynucleotide of the first probe is between the analyte binding domain and the ligand (or solid support) and the 3′ end is not available for extension, then the polynucleotide from the second probe is the only polynucleotide extended while using the polynucleotide of the first probe as the template.

In some aspects, the method can further comprise amplifying the extended polynucleotides. In some aspects, the extension product can be amplified through PCR. In some aspects, the extension product is amplified, forming an amplified product, and appended with sequences used for downstream DNA sequencing and error correction (UMIs).

In some aspects, the method can further comprise sequencing the polynucleotides to generate sequencing reads. Any method of sequencing can be used with the disclosed methods. For example, the disclosed methods can also include Sanger sequencing (chain termination method), high-throughput sequencing (NGS), whole genome sequencing, shotgun sequencing, Maxam-Gilbert sequencing, capillary electrophoresis, methylation sequencing, or RNA sequencing (whole transcriptome sequencing). In some aspects, the sequence reads can indicate the presence or amount of a specific target analyte. For example, in some aspects, the number of molecules of specific target analyte is determined bioinformatically and related to a suitable reference or standard curve acquired contemporaneously with the assay. The sequencing can be performed after amplifying the extended polynucleotides. In some aspects, the amplified product can be purified and sequenced by NGS on a suitable platform. In some aspects, any sequencing technology can be used. In some aspects, sequencing instruments can include Illumina instruments like MiSeq, NextSeq, NovaSeq, MiniSeq, HiSeq, HiSeq X, HiSeq 3000/4000, and HiSeq 2500. Oxford Nanopore Technologies (ONT) instruments can include MinION, GridION, PromethION, and Flongle. Singular genomics instruments include the G4, and PX sequencing platforms. Element Biosciences platforms include AVITI.

In some aspects, the method can further comprise immobilizing the first complex after contacting the first complex with a capture molecule and before contacting the captured first complex with at least one second probe. In some aspects, immobilizing can be dependent on what capture molecule and solid support is used. For example, if the solid support is a magnetic bead bound to a capture molecule, streptavidin, then a magnet can be used to immobilize the captured first complex which comprises the target analyte bound to the analyte binding domain of the first probe and the capture molecule (bound to a solid support) bound to the ligand of the first probe. In some aspects, if the solid support is an agarose or sepharose bead, immobilization can comprise using a column that holds the beads and anything bound/associated with the beads.

In some aspects, the sample further comprises one or more non-target analytes. In some aspects, the methods further comprise removing the one or more non-target analytes from the sample. In some aspects, removing the one or more non-target analytes comprises the step of immobilizing the first complex after contacting the first complex with a capture molecule and before contacting the captured first complex with at least one second probe. In some aspects, the immobilization allows for the captured first complex to remain immobilized while the non-target analytes from the sample are removed, or washed away.

In some aspects, immobilization allows for washing or removing any unbound second probe. Thus, the methods can further comprise removing any unbound second probes after immobilizing and after contacting the captured first complex with at least one second probe. In some aspects, any unbound second probe will not be immobilized as part of the second complex.

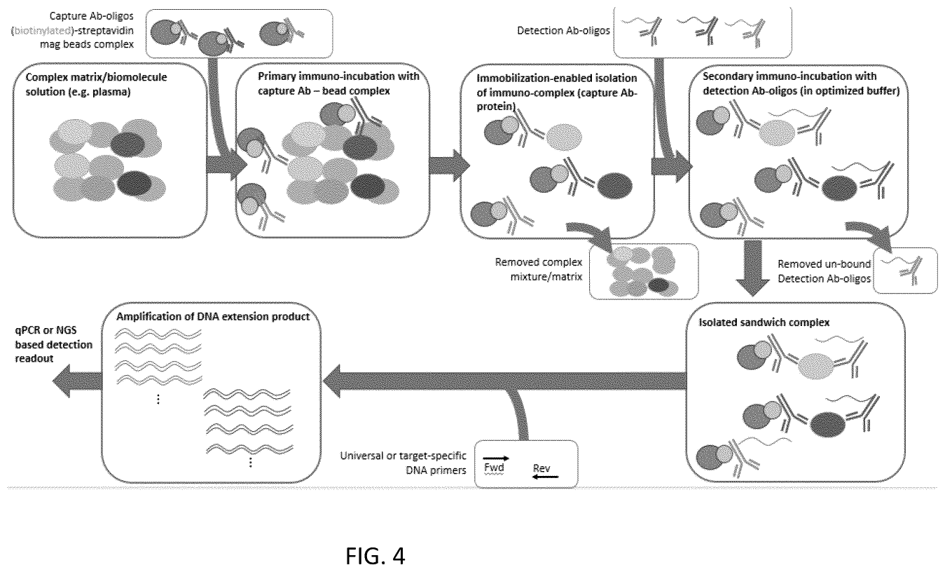

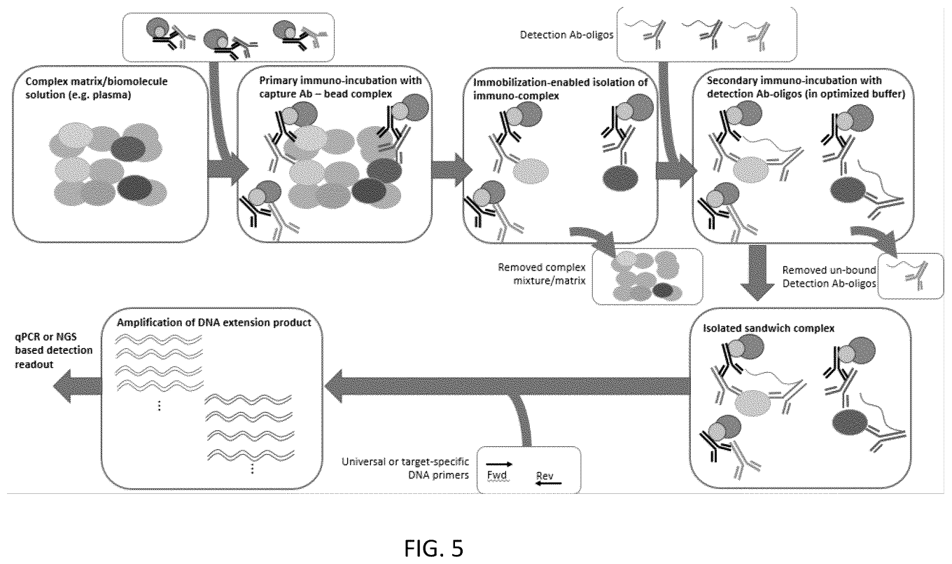

In some aspects, the methods can include the assay as shown in FIGS. 4 and 5. For example, in some aspects only one of either the first probe or second probe comprises a polynucleotide. When using this technique, primers specific to the polynucleotide can be added to generate amplification products since there is no complementary binding of polynucleotides between the first probe and second probe.

i. First Probe

In some aspects, the first probe comprises a ligand, an analyte binding domain and a polynucleotide. In some aspects, the order of the ligand, analyte binding domain and polynucleotide can vary.

In some aspects, the ligand acts as a binding partner to the capture molecule. For example, in some aspects, the ligand can be, but is not limited to, biotin or a biotin analogue, protein A/G, protein L. Other examples may include His tag/nickel, GST tag/GSH, SpyTag/SpyCatcher, Maltose Binding Protein/Amylose, FLAG/anti-FLAG Ab, FITC/anti-FITC Ab, and digoxigenin/anti-digoxigenin. Furthermore, the binding partner may be linked to the capture molecule and polynucleotide through direct covalent linkage. The covalent linkage may be formed through conjugation chemistry known to those skilled in the art, such as carbodiimide-mediated amine-carboxylic acid amide formation, click chemistry, maleimide-thiol, Diels-Alder reaction, azide-alkyne cyclo addition, etc.

In some aspects, the analyte binding domain of the first probe can be anything that binds to the specific target analyte. For example, the analyte binding domain can be an antibody, or antigen binding fragment thereof, capable of binding to the specific target analyte. Thus, the analyte binding domain of the first probe can be a target analyte specific antibody. In some aspects, the analyte binding domain can be, but is not limited to a protein, peptide, or nucleic acid sequence known to bind the specific target analyte.

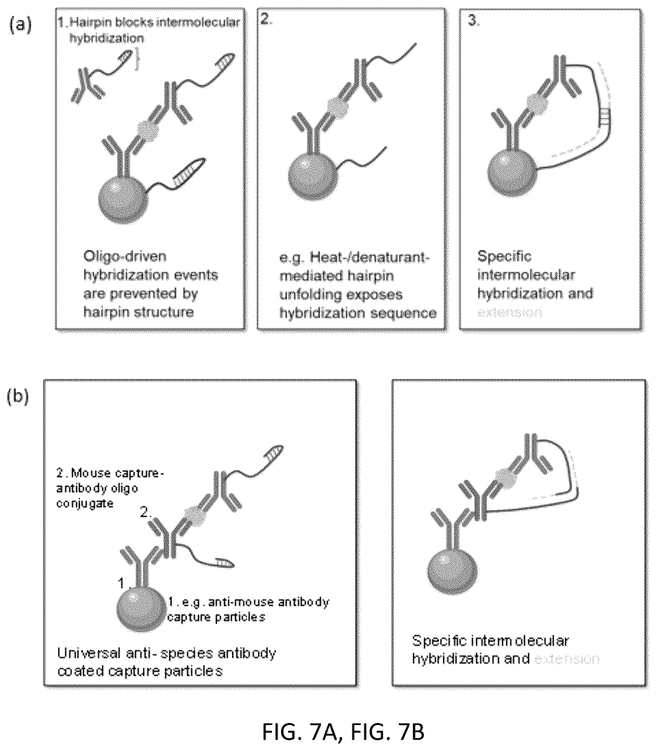

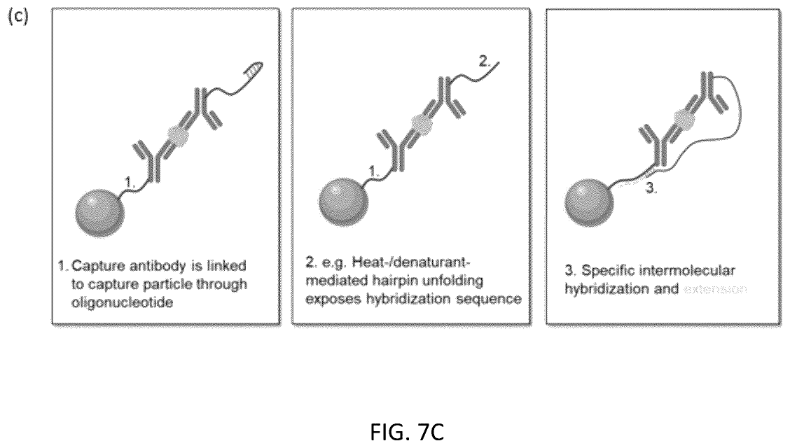

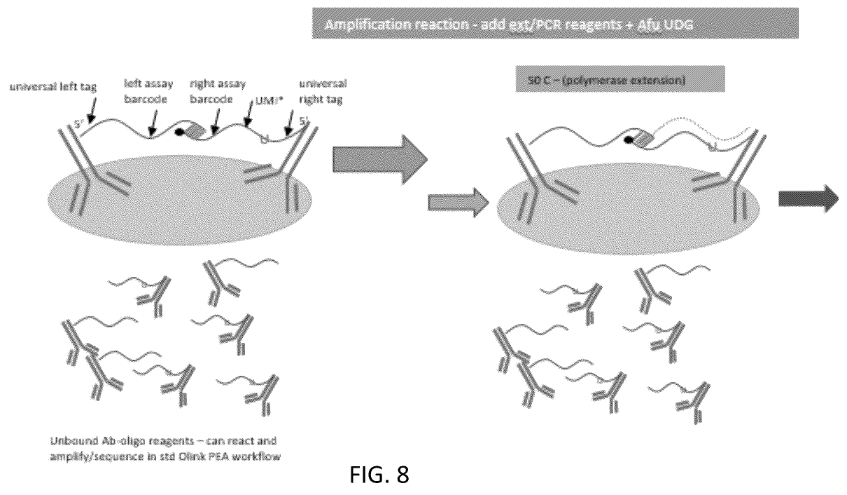

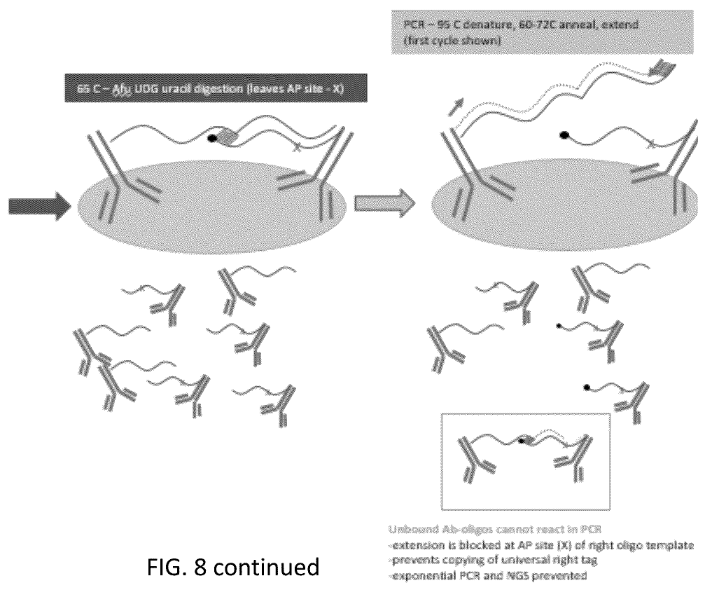

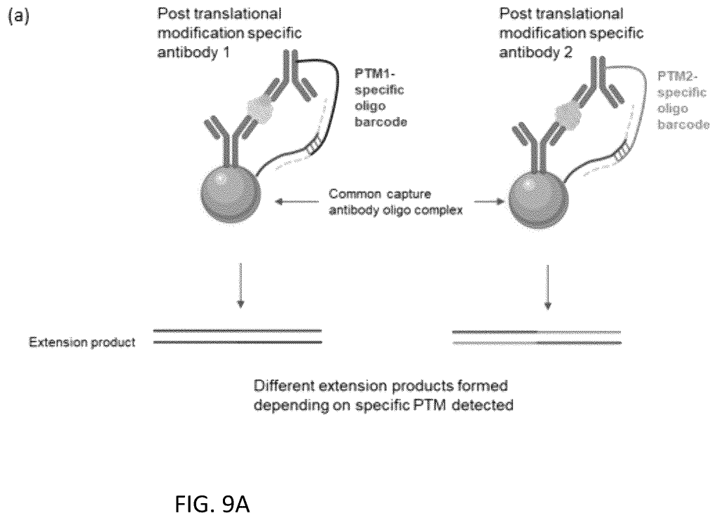

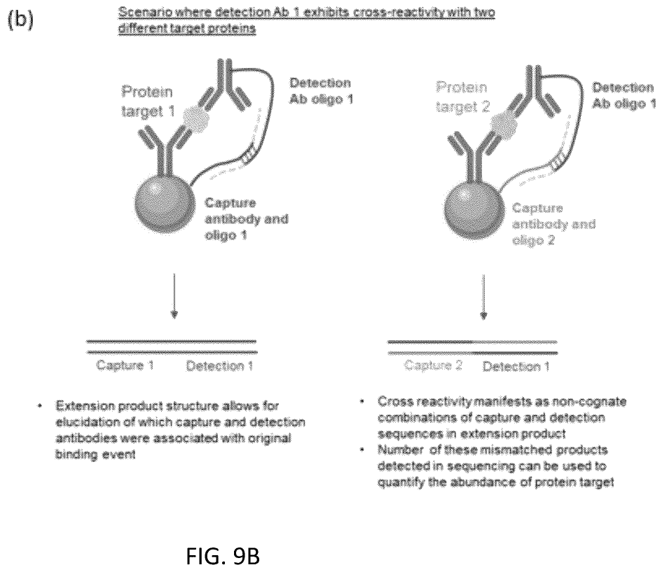

In some aspects, the polynucleotide of the first probe comprises a sequence complementary to a portion of the polynucleotide of the second probe. In some aspects, the polynucleotide of the first probe can be bound to the ligand of the first probe. In some aspects, the polynucleotide of the first probe can be bound to the analyte binding domain of the first probe. In some aspects, the polynucleotide of the first probe can be attached on its 5′ end thus allowing extension from the 3′ end. In some aspects, the polynucleotide of the first probe can be bound to the ligand and the analyte binding domain. Examples of where the polynucleotide can be bound in the first probe can be seen in FIG. 7. In some aspects, the polynucleotide can further comprise a blocking oligonucleotide, an examples of which is shown in FIGS. 4 and 7. In some aspects, the polynucleotide of the first probe comprises a barcode sequence. In some aspects, the barcode sequence is specifically associated with a target analyte. In some aspects, the barcodes can sometimes be referred to as unique molecular identifiers (or UMIs). Unique molecular identifiers (UMIs) are a method for counting molecules in single-cell RNA sequencing (scRNA-seq). UMIs can be random sequences added to DNA before amplification and sequencing to distinguish between molecules within a cell. The barcode can be used for PCR/sequencing error correction via molecular counting. Methods for molecular counting using UMIs are known in the art and can include, but are not limited to UMI method, TRUmiCount, DAUMI and AmpliCI.

ii. Second Probe

In some aspects, the second probe comprises an analyte binding domain specific for a target analyte and a polynucleotide.

In some aspects, the analyte binding domain of the second probe can be specific for the target analyte in the captured first complex. In some aspects, the analyte binding domain of the second probe can be anything that binds to the specific target analyte. For example, the analyte binding domain can be an antibody, or antigen binding fragment thereof, known to bind the specific target analyte. Thus, the analyte binding domain of the first probe can be a target analyte specific antibody. In some aspects, the analyte binding domain can be, but is not limited to a protein, peptide, or nucleic acid sequence known to bind the specific target analyte.

In some aspects, the analyte binding domain of the second probe can bind to a different epitope of the specific target analyte than the analyte binding domain of the first probe.

In some aspects, the polynucleotide of the second probe comprises a sequence complementary to a portion of the polynucleotide of the first probe. In some aspects, the polynucleotide of the second probe can be bound to the analyte binding domain of the second probe. In some aspects, the polynucleotide of the second probe can be attached on its 5′ end thus allowing extension from the 3′ end. In some aspects, the polynucleotide can further comprise a blocking oligonucleotide, examples of which are shown in FIG. 7.

iii. Capture Molecule

In some aspects, the capture molecule can be anything that binds to the ligand of the first probe. The capture molecule can be, but is not limited to streptavidin and other biotin-binding proteins (binds biotin or biotin analogues), Fc region of immunoglobulins (binds protein A/G), immunoglobulin (Ig) kappa light chains (binds protein L).

In some aspects, the capture molecule is bound to a solid support, thereby immobilizing the captured first complex. Thus, in some aspects, when contacting the first complex with a capture molecule, the capture molecule is already bound to a solid support. In some aspects, a solid support can be a magnetic substance, such as, but not limited to a magnetic particle or a magnetic bead. In some aspects, a solid support can be protein A/G agarose beads, protein A/G sepharose beads, protein L beads, streptavidin or avidin beads, protein magnetic microplates, protein capture resins, nanobodies, cellulose beads, monoclonal antibodies, antigen-purified antibodies. In some embodiments, the solid support comprises a bead. In some embodiments, the bead is selected from the group consisting of: silica gel bead, controlled pore glass bead, magnetic bead, Dynabead, Sephadex/Sepharose beads, cellulose beads, and polystyrene beads, or any combination thereof. In some embodiments, the bead comprises a magnetic bead. In some embodiments, the solid support is semi-solid. In some embodiments, the solid support comprises a polymer, a matrix, or a hydrogel. In some embodiments, the solid support comprises a needle array device.

In some aspects, referring to the capture molecule or the solid support can also refer to the combination of the capture molecule and the solid support as they can be used together to immobilize the target analyte.

In some aspects, the capture molecule can be an antibody that is specific to the one or more target analytes. In some aspects, when the capture molecule is an antibody, the solid support can be any of the solid supports described herein bound to an anti-IgG antibody. Thus, the anti-IgG antibody can bind to the capture molecule (e.g., antibody specific to the target analyte) thus forming a captured first complex. An example can be seen in FIG. 5.

iv. Blocking Hybridization

In some aspects, the methods further comprise blocking the hybridization of the polynucleotide of the first and second probes from occurring too soon. False-positives can result if the polynucleotide of the first and second probes hybridize before the second complex is formed (for example, in the absence of target analyte). In some aspects, hybridization can be blocked using any known techniques, such as, but not limited to, using a blocking oligonucleotide, incorporating a hairpin loop into one or both polynucleotides of the first or second probe, adding a blocking reagent to the reaction mixture or adding In situ hybridization (ISH) blockers to the reaction mixture.

In some aspects, the polynucleotide of the first probe, second probe, or both can further comprise a blocking oligonucleotide. In some aspects, the blocking oligonucleotide is complementary to a portion of the polynucleotide of the first probe. In some aspects, the blocking oligonucleotide is complementary to a portion of the polynucleotide of the second probe. In some aspects, there are two blocking oligonucleotides, wherein one is complementary to a portion of the polynucleotide of the first probe and one is complementary to a portion of the polynucleotide of the second probe.

In some aspects, the blocking oligonucleotide hybridizes to a portion of the polynucleotide of the first or second probe it is complementary to. In some aspects, the complementary portion, and thus the hybridization of the blocking oligonucleotide, is at the 3′ end of the polynucleotide of the first or second probe. Because extension can occur at the 3′ end of the polynucleotide the presence of the blocking oligonucleotide hybridized at the 3′ end of the polynucleotide of the first or second probe can prevent extension until the exact time needed.

In some aspects, the polynucleotide of the first probe, second probe, or both can further comprise a hairpin loop at the 3′ end of the polynucleotide. In some aspects, a hairpin loop is a secondary structure formed in the polynucleotide of the first probe, second probe, or both where a section of the polynucleotide folds back on itself to form base pairs with a complementary sequence within the polynucleotide. In some aspects, the polynucleotide of the first probe, second probe, or both comprises a first sequence complementary to a second sequence of the same polynucleotide, thus allowing it to hybridize with itself.

In some aspect of the methods involving the blocking oligonucleotide or hairpin loop, the methods can further comprise removing the blocking oligonucleotide or hairpin loop prior to incubating the second complex under conditions to allow for hybridization of the polynucleotide of the first probe to the polynucleotide of the second probe. In some aspects, removing the blocking oligonucleotide or hairpin loop comprises heat, enzymatic digestion, or introduction of a denaturant. For example, the method can comprise increasing the temperature of the reaction to allow denaturation of the blocking oligonucleotide or hairpin loop.

In some aspects, the blocking oligonucleotide comprises uracil residues dispersed throughout the sequence. Therefore, in some aspects, removing the blocking oligonucleotide can comprise uracil-specific endonuclease digestion. In some aspects, a similar strategy can be used for the hairpin loop.

### 2. Bind Capture Molecule to First Probe Before Contacting to Target Analyte

Also disclosed herein are methods wherein the first probe comprises a capture molecule wherein the first probe is bound to a capture molecule after the first probe is contacted to the sample comprising one or more target analytes. In some aspects, all of the steps starting with contacting the captured first complex with at least one second probe, and all of the steps thereafter, are the same between the two methods.

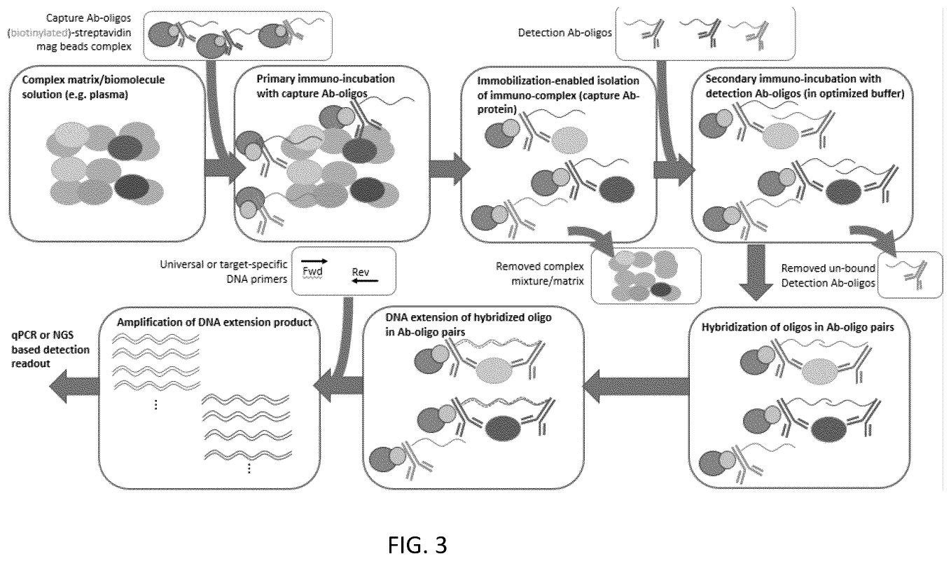

Additional aspects of the disclosed methods are illustrated in FIG. 3 and FIG. 4. A broader description of the method is as follows.

Disclosed are methods of contacting a sample comprising one or more target analytes with at least one first probe, wherein the first probe comprises a capture molecule, a ligand, an analyte binding domain and a polynucleotide, wherein the ligand is bound to the capture molecule, wherein the first probe specifically binds to one of the target analytes in the sample, thereby generating a captured first complex comprising the target analyte bound to the analyte binding domain of the first probe; contacting the captured first complex with at least one second probe, wherein the second probe comprises an analyte binding domain specific for the target analyte in the captured first complex and a polynucleotide, wherein the polynucleotide of the second probe comprises a sequence complementary to a portion of the polynucleotide of the first probe, wherein the second probe specifically binds to the target analyte in the captured first complex, thereby generating at least one second complex; incubating the second complex under conditions to allow for hybridization of the polynucleotide of the first probe to the polynucleotide of the second probe; and extending the hybridized polynucleotides of the first and second probes to generate complementary strands of the polynucleotides of the first and second probes.

In some aspects, incubating the second complex under conditions to allow for hybridization of the polynucleotide of the first probe to the polynucleotide of the second probe at the complementary sequences of each. In some aspects, the result is a duplex of the polynucleotide of the first probe to the polynucleotide of the second probe. In some aspects, the 3′ end of the polynucleotide of the second probe hybridizes to the 3′ end of the polynucleotide of the first probe. Thus, in some aspects, the sequence of the second probe that is complementary to a portion of the polynucleotide of the first probe is present at the 3′ end of the polynucleotide of the second probe.

In some aspects, extending the hybridized polynucleotides of the first and second probes to generate complementary strands of the polynucleotides of the first and second probes comprises incubating the at least one second complex having hybridized polynucleotides with a solution comprising at least an extending polymerase and nucleotides allowing extension of the hybridized polynucleotides. In some aspects, the extension can be performed identical to the extension step of known proximity extension assays. In some aspects, one or both of the polynucleotides of the first and second probes are extended. For example, if the polynucleotide of the first probe is between the analyte binding domain and the ligand (or solid support) and the 3′ end is not available for extension, then the polynucleotide from the second probe is the only polynucleotide extended while using the polynucleotide of the first probe as the template.

In some aspects, the method can further comprise amplifying the extended polynucleotides. In some aspects, the extension product can be amplified through PCR. In some aspects, the extension product is amplified, forming an amplified product, and appended with sequences used for downstream DNA sequencing and error correction (UMIs).

In some aspects, the method can further comprise sequencing the polynucleotides to generate sequencing reads. Any method of sequencing can be used with the disclosed methods. For example, the disclosed methods can also include Sanger sequencing (chain termination method), high-throughput sequencing (NGS), whole genome sequencing, shotgun sequencing, Maxam-Gilbert sequencing, capillary electrophoresis, methylation sequencing, or RNA sequencing (whole transcriptome sequencing). In some aspects, the sequence reads can indicate the presence or amount of a specific target analyte. For example, in some aspects, the number of molecules of specific target analyte is determined bioinformatically and related to a suitable reference or standard curve acquired contemporaneously with the assay. The sequencing can be performed after amplifying the extended polynucleotides. In some aspects, the amplified product can be purified and sequenced by NGS on a suitable platform. In some aspects, any sequencing technology can be used. In some aspects, sequencing instruments can include Illumina instruments like MiSeq, NextSeq, NovaSeq, MiniSeq, HiSeq, HiSeq X, HiSeq 3000/4000, and HiSeq 2500. Oxford Nanopore Technologies (ONT) instruments can include MinION, GridION, PromethION, and Flongle. Singular genomics instruments include the G4, and PX sequencing platforms. Element Biosciences platforms include AVITI.

In some aspects, the sample further comprises one or more non-target analytes. In some aspects, the methods further comprise removing the one or more non-target analytes from the sample. In some aspects, removing the one or more non-target analytes comprises the step of immobilizing which comprises the captured first complex that is bound to a solid support. In some aspects, the immobilization allows for the captured first complex to remain immobilized while the non-target analytes from the sample are removed, or washed away.

In some aspects, immobilization allows for washing or removing any unbound second probe. Thus, the methods can further comprise removing any unbound second probes after contacting the captured first complex with at least one second probe. In some aspects, any unbound second probe will not be immobilized as part of the second complex.

As shown in FIGS. 4 and 5, in some aspects only one of either the first probe or second probe comprises a polynucleotide. When using this technique, primers specific to the polynucleotide can be added to generate amplification products since there is no complementary binding of polynucleotides between the first probe and second probe.

i. First Probe

In some aspects, the first probe comprises a capture molecule, a ligand, an analyte binding domain and a polynucleotide. In some aspects, the order of the capture molecule, ligand, analyte binding domain and polynucleotide can vary.

In some aspects, the capture molecule can be anything that binds to the ligand of the first probe. The capture molecule can be, but is not limited to streptavidin and other biotin-binding proteins (binds biotin or biotin analogues), Fc region of immunoglobulins (binds protein A/G), or immunoglobulin (Ig) kappa light chains (binds protein L).

In some aspects, the capture molecule is bound to a solid support, thereby immobilizing a first complex and creating a captured first complex. In some aspects, referring to the capture molecule or the solid support can refer to the combination of the capture molecule and the solid support as they can be used together to immobilize the target analyte. Thus, in some aspects, when contacting the sample with the first probe, the capture molecule is already bound to a solid support. In some aspects, a solid support can be a magnetic substance, such as, but not limited to a magnetic particle or a magnetic bead. In some aspects, a solid support can be protein A/G agarose beads, protein A/G sepharose beads, protein L beads, streptavidin or avidin beads, protein magnetic microplates, protein capture resins, nanobodies, cellulose beads, monoclonal antibodies, antigen-purified antibodies. In some embodiments, the solid support comprises a bead. In some embodiments, the bead is selected from the group consisting of: silica gel bead, controlled pore glass bead, magnetic bead, Dynabead, Sephadex/Sepharose beads, cellulose beads, and polystyrene beads, or any combination thereof. In some embodiments, the bead comprises a magnetic bead. In some embodiments, the solid support is semi-solid. In some embodiments, the solid support comprises a polymer, a matrix, or a hydrogel. In some embodiments, the solid support comprises a needle array device.

In some aspects, the capture molecule can be an antibody that is specific to the one or more target analytes. In some aspects, when the capture molecule is an antibody, the solid support can be any of the solid supports described herein bound to an anti-IgG antibody. Thus, the anti-IgG antibody can bind to the capture molecule (e.g., antibody specific to the target analyte) thus forming a captured first complex. An example of this can be seen in FIG. 5.

In some aspects, the ligand acts as a binding partner to the capture molecule. For example, in some aspects, the ligand can be, but is not limited to, biotin or a biotin analogue, protein A/G, protein L. In some aspects, the ligand acts as a binding partner to the capture molecule. For example, in some aspects, the ligand can be, but is not limited to, biotin or a biotin analogue, protein A/G, protein L. Other examples may include His tag/nickel, GST tag/GSH, SpyTag/SpyCatcher, Maltose Binding Protein/Amylose, FLAG/anti-FLAG Ab, FITC/anti-FITC Ab, and digoxigenin/anti-digoxigenin. Furthermore, the binding partner may be linked to the capture molecule and polynucleotide through direct covalent linkage. The covalent linkage may be formed through conjugation chemistry known to those skilled in the art, such as carbodiimide-mediated amine-carboxylic acid amide formation, click chemistry, maleimide-thiol, Diels-Alder reaction, azide-alkyne cyclo addition, etc.

In some aspects, the ligand interacts with the capture molecule and another portion of the first probe such as the analyte binding domain or polynucleotide.

In some aspects, the first probe does not comprise a ligand but rather has a solid support bound directly to the analyte binding domain or polynucleotide of the first probe. Thus, in some aspects, the first probe comprises a solid support, an analyte binding domain and a polynucleotide. In some aspects, the polynucleotide of the first probe can be bound to the solid support of the first probe.

In some aspects, the analyte binding domain of the first probe can be the same as the analyte binding domain of the first probe described in the method above. In some aspects, the analyte binding domain of the first probe can be anything that binds to the specific target analyte. For example, the analyte binding domain can be an antibody, or antigen binding fragment thereof, known to bind the specific target analyte. Thus, the analyte binding domain of the first probe can be a target analyte specific antibody. In some aspects, the analyte binding domain can be, but is not limited to a protein, peptide, or nucleic acid sequence known to bind the specific target analyte.

In some aspects, the polynucleotide of the first probe comprises a sequence complementary to a portion of the polynucleotide of the second probe. In some aspects, the polynucleotide of the first probe can be bound to the ligand of the first probe. In some aspects, the polynucleotide of the first probe can be bound to the analyte binding domain of the first probe. In some aspects, the polynucleotide of the first probe can be attached on its 5′ end thus allowing extension from the 3′ end. In some aspects, the polynucleotide of the first probe can be bound to the ligand and the analyte binding domain. Examples of where the polynucleotide can be bound in the first probe can be seen in FIG. 7. In some aspects, the polynucleotide can further comprise a blocking oligonucleotide, an example of which is shown in FIGS. 4 and 7. In some aspects, the polynucleotide of the first probe comprises a barcode sequence. In some aspects, the barcode sequence is specifically associated with a target analyte. In some aspects, the barcodes can sometimes be referred to as unique molecular identifiers (or UMIs). Unique molecular identifiers (UMIs) are a method for counting molecules in single-cell RNA sequencing (scRNA-seq). UMIs can be random sequences added to DNA before amplification and sequencing to distinguish between molecules within a cell. The barcode can be used for PCR/sequencing error correction via molecular counting. Methods for molecular counting using UMIs are known in the art and can include, but are not limited to UMI method, TRUmiCount, DAUMI and AmpliCI.

ii. Second Probe

In some aspects, the second probe can be identical to the second probe used in the above methods. In some aspects, the second probe comprises an analyte binding domain specific for a target analyte and a polynucleotide.

In some aspects, the analyte binding domain of the second probe can be specific for the target analyte in the captured first complex. In some aspects, the analyte binding domain of the second probe can be anything that binds to the specific target analyte. For example, the analyte binding domain can be an antibody, or antigen binding fragment thereof, known to bind the specific target analyte. Thus, the analyte binding domain of the first probe can be a target analyte specific antibody. In some aspects, the analyte binding domain can be, but is not limited to a protein, peptide, or nucleic acid sequence known to bind the specific target analyte.

In some aspects, the analyte binding domain of the second probe can bind to a different epitope of the specific target analyte than the analyte binding domain of the first probe.

In some aspects, the polynucleotide of the second probe comprises a sequence complementary to a portion of the polynucleotide of the first probe. In some aspects, the polynucleotide of the second probe can be bound to the analyte binding domain of the second probe. In some aspects, the polynucleotide of the second probe can be attached on its 5′ end thus allowing extension from the 3′ end. In some aspects, the polynucleotide can further comprise a blocking oligonucleotide, examples of which are shown in FIGS. 4 and 7.

In some aspects, the ligand acts as a binding partner to the capture molecule. For example, in some aspects, the ligand can be, but is not limited to, biotin or a biotin analogue, protein A/G, protein L. In some aspects, the ligand acts as a binding partner to the capture molecule. For example, in some aspects, the ligand can be, but is not limited to, biotin or a biotin analogue, protein A/G, protein L. Other examples may include His tag/nickel, GST tag/GSH, SpyTag/SpyCatcher, Maltose Binding Protein/Amylose, FLAG/anti-FLAG Ab, FITC/anti-FITC Ab, and digoxigenin/anti-digoxigenin. Furthermore, the binding partner may be linked to the capture molecule and polynucleotide through direct covalent linkage. The covalent linkage may be formed through conjugation chemistry known to those skilled in the art, such as carbodiimide-mediated amine-carboxylic acid amide formation, click chemistry, maleimide-thiol, Diels-Alder reaction, azide-alkyne cyclo addition, etc.

In some aspects, the analyte binding domain of the second probe can be the same as the analyte binding domain of the second probe of the method described above. In some aspects, the analyte binding domain of the second probe can be specific for the target analyte in the captured first complex. In some aspects, the analyte binding domain of the second probe can be anything that binds to the specific target analyte. For example, the analyte binding domain can be an antibody, or antigen binding fragment thereof, known to bind the specific target analyte. Thus, the analyte binding domain of the first probe can be a target analyte specific antibody. In some aspects, the analyte binding domain can be, but is not limited to a protein, peptide, or nucleic acid sequence known to bind the specific target analyte.

In some aspects, the analyte binding domain of the second probe can bind to a different epitope of the specific target analyte than the analyte binding domain of the first probe.

In some aspects, the polynucleotide of the first probe comprises a sequence complementary to a portion of the polynucleotide of the second probe. In some aspects, the polynucleotide of the first probe can be bound to the ligand of the first probe. In some aspects, the polynucleotide of the first probe can be bound to the analyte binding domain of the first probe. In some aspects, the polynucleotide of the first probe can be attached on its 5′ end thus allowing extension from the 3′ end. In some aspects, the polynucleotide of the first probe can be bound to the ligand and the analyte binding domain. Examples of where the polynucleotide can be bound in the first probe can be seen in FIG. 7. In some aspects, the polynucleotide can further comprise a blocking oligonucleotide, examples of which are shown in FIGS. 4 and 7. In some aspects, the polynucleotide of the first probe comprises a barcode sequence. In some aspects, the barcode sequence is specifically associated with a target analyte. In some aspects, the barcodes can sometimes be referred to as unique molecular identifiers (or UMIs). The barcode can be used for PCR/sequencing error correction via molecular counting. Methods for molecular counting using UMIs are known in the art.

iii. Blocking Hybridization

In some aspects, the methods further comprise blocking the hybridization of the polynucleotide of the first and second probes from occurring too soon the same as described for the above method. False-positives can result if the polynucleotide of the first and second probes hybridize before the second complex is formed (for example, in the absence of target analyte). In some aspects, hybridization can be blocked using any known techniques, such as, but not limited to, using a blocking oligonucleotide, incorporating a hairpin loop into one or both polynucleotides of the first or second probe, adding a blocking reagent to the reaction mixture or adding In situ hybridization (ISH) blockers to the reaction mixture.

In some aspects, the polynucleotide of the first probe, second probe, or both can further comprise a blocking oligonucleotide. In some aspects, the blocking oligonucleotide is complementary to a portion of the polynucleotide of the first probe. In some aspects, the blocking oligonucleotide is complementary to a portion of the polynucleotide of the second probe. In some aspects, there are two blocking oligonucleotides, wherein one is complementary to a portion of the polynucleotide of the first probe and one is complementary to a portion of the polynucleotide of the second probe.

In some aspects, the blocking oligonucleotide hybridizes to a portion of the polynucleotide of the first or second probe to which it is complementary. In some aspects, the complementary portion, and thus the hybridization of the blocking oligonucleotide, is at the 3′ end of the polynucleotide of the first or second probe. Because extension can occur at the 3′ end of the polynucleotide the presence of the blocking oligonucleotide hybridized at the 3′ end of the polynucleotide of the first or second probe can prevent extension until the exact time needed.

In some aspects, the polynucleotide of the first probe, second probe, or both can further comprise a hairpin loop at the 3′ end of the polynucleotide. In some aspects, a hairpin loop is a secondary structure formed in the polynucleotide of the first probe, second probe, or both where a section of the polynucleotide folds back on itself to form base pairs with a complementary sequence within the polynucleotide. In some aspects, the polynucleotide of the first probe, second probe, or both comprises a first sequence complementary to a second sequence of the same polynucleotide, thus allowing it to hybridize with itself.

In some aspect of the methods involving the blocking oligonucleotide or hairpin loop, the methods can further comprise removing the blocking oligonucleotide or hairpin loop prior to incubating the second complex under conditions to allow for hybridization of the polynucleotide of the first probe to the polynucleotide of the second probe. In some aspects, removing the blocking oligonucleotide or hairpin loop comprises heat, enzymatic digestion, or introduction of a denaturant. For example, the method can comprise increasing the temperature of the reaction to allow denaturation of the blocking oligonucleotide or hairpin loop.

In some aspects, the blocking oligonucleotide comprises uracil residues dispersed throughout the sequence. Therefore, in some aspects, removing the blocking oligonucleotide can comprise uracil-specific endonuclease digestion. In some aspects, a similar strategy can be used for the hairpin loop.

### 3. Direct Conjugation of Solid Support to First Probe, No Ligand Needed

The method of direct conjugation of a solid support to a first probe is very similar to the methods described above exact no ligand is present on the first probe and a solid support is directly conjugated to the first probe. In fact, in some aspects, all of the steps starting with contacting the captured first complex with at least one second probe, and all of the steps thereafter, are the same between the two methods.

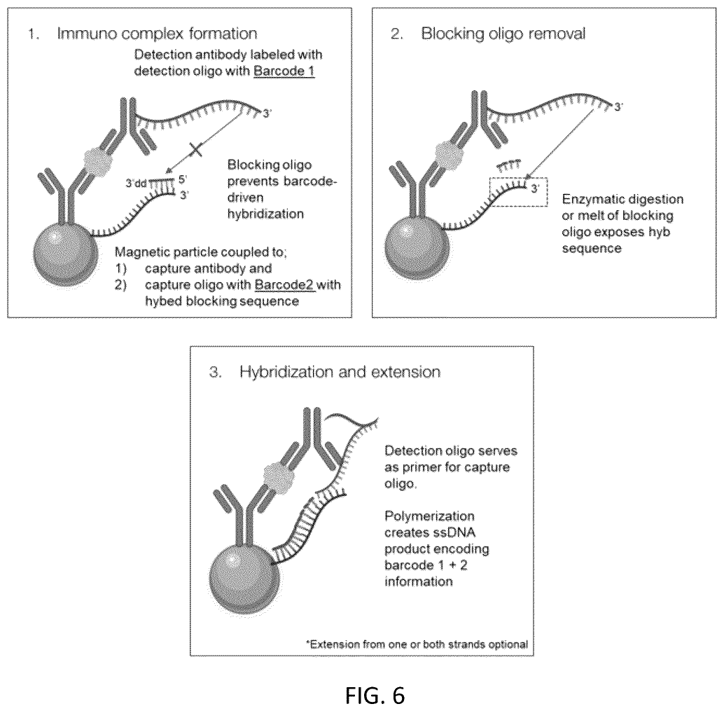

An example of the disclosed methods is illustrated in FIG. 6. A broader description of the method is as follows.

Disclosed are methods of contacting a sample comprising one or more target analytes with at least one first probe, wherein the first probe comprises a capture molecule, an analyte binding domain and a polynucleotide, wherein the first probe specifically binds to one of the target analytes in the sample, thereby generating a captured first complex comprising the target analyte bound to the analyte binding domain of the first probe; contacting the captured first complex with at least one second probe, wherein the second probe comprises an analyte binding domain specific for the target analyte in the captured first complex and a polynucleotide, wherein the polynucleotide of the second probe comprises a sequence complementary to a portion of the polynucleotide of the first probe, wherein the second probe specifically binds to the target analyte in the captured first complex, thereby generating at least one second complex; incubating the second complex under conditions to allow for hybridization of the polynucleotide of the first probe to the polynucleotide of the second probe; and extending the hybridized polynucleotides of the first and second probes to generate complementary strands of the polynucleotides of the first and second probes.

In some aspects, incubating the second complex under conditions to allow for hybridization of the polynucleotide of the first probe to the polynucleotide of the second probe at the complementary sequences of each. In some aspects, the result is a duplex of the polynucleotide of the first probe to the polynucleotide of the second probe. In some aspects, the 3′ end of the polynucleotide of the second probe hybridizes to the 3′ end of the polynucleotide of the first probe. Thus, in some aspects, the sequence of the second probe that is complementary to a portion of the polynucleotide of the first probe is present at the 3′ end of the polynucleotide of the second probe.

In some aspects, extending the hybridized polynucleotides of the first and second probes to generate complementary strands of the polynucleotides of the first and second probes comprises incubating the at least one second complex having hybridized polynucleotides with a solution comprising at least an extending polymerase and nucleotides allowing extension of the hybridized polynucleotides. In some aspects, the extension can be performed under the same or similar conditions as the extension step of known proximity extension assays. In some aspects, one or both of the polynucleotides of the first and second probes are extended. For example, if the polynucleotide of the first probe is between the analyte binding domain and the ligand (or solid support) and the 3′ end is not available for extension, then the polynucleotide from the second probe is the only polynucleotide extended while using the polynucleotide of the first probe as the template.

In some aspects, the method can further comprise amplifying the extended polynucleotides. In some aspects, the extension product can be amplified through PCR. In some aspects, the extension product is amplified, forming an amplified product, and appended with sequences used for downstream DNA sequencing and error correction (UMIs).

In some aspects, the method can further comprise sequencing the polynucleotides to generate sequencing reads. Any method of sequencing can be used with the disclosed methods. For example, the disclosed methods can also include Sanger sequencing (chain termination method), high-throughput sequencing (NGS), whole genome sequencing, shotgun sequencing, Maxam-Gilbert sequencing, capillary electrophoresis, methylation sequencing, or RNA sequencing (whole transcriptome sequencing). In some aspects, the sequence reads can indicate the presence or amount of a specific target analyte. For example, in some aspects, the number of molecules of specific target analyte is determined bioinformatically and related to a suitable reference or standard curve acquired contemporaneously with the assay. The sequencing can be performed after amplifying the extended polynucleotides. In some aspects, the amplified product can be purified and sequenced by NGS on a suitable platform. In some aspects, any sequencing technology can be used. In some aspects, sequencing instruments can include Illumina instruments like MiSeq, NextSeq, NovaSeq, MiniSeq, HiSeq, HiSeq X, HiSeq 3000/4000, and HiSeq 2500. Oxford Nanopore Technologies (ONT) instruments can include MinION, GridION, PromethION, and Flongle. Singular genomics instruments include the G4, and PX sequencing platforms. Element Biosciences platforms include AVITI.

In some aspects, the sample further comprises one or more non-target analytes. In some aspects, the methods further comprise removing the one or more non-target analytes from the sample. In some aspects, removing the one or more non-target analytes comprises the step of immobilizing which comprises the captured first complex that is bound to a solid support. In some aspects, the immobilization allows for the captured first complex to remain immobilized while the non-target analytes from the sample are removed, or washed away.

In some aspects, immobilization allows for washing or removing any unbound second probe. Thus, the methods can further comprise removing any unbound second probes after contacting the captured first complex with at least one second probe. In some aspects, any unbound second probe will not be immobilized as part of the second complex.

In some aspects, the methods can include the assay as shown in FIGS. 4 and 5. For example, in some aspects only one of either the first probe or second probe comprises a polynucleotide. When using this technique, primers specific to the polynucleotide can be added to generate amplification products since there is no complementary binding of polynucleotides between the first probe and second probe.

i. First Probe

In some aspects, the first probe comprises a solid support, an analyte binding domain and a polynucleotide. In some aspects, the order of the solid support, analyte binding domain and polynucleotide can vary.

In some aspects, a solid support can be a magnetic substance, such as, but not limited to a magnetic particle or a magnetic bead. In some aspects, a solid support can be protein A/G agarose beads, protein A/G sepharose beads, protein L beads, streptavidin or avidin beads, protein magnetic microplates, protein capture resins, nanobodies, cellulose beads, monoclonal antibodies, antigen-purified antibodies. In some embodiments, the solid support comprises a bead. In some embodiments, the bead is selected from the group consisting of: silica gel bead, controlled pore glass bead, magnetic bead, Dynabead, Sephadex/Sepharose beads, cellulose beads, and polystyrene beads, or any combination thereof. In some embodiments, the bead comprises a magnetic bead. In some embodiments, the solid support is semi-solid. In some embodiments, the solid support comprises a polymer, a matrix, or a hydrogel. In some embodiments, the solid support comprises a needle array device.

In some aspects, the analyte binding domain of the first probe can be the same as the analyte binding domain of the first probe described in the method above. In some aspects, the analyte binding domain of the first probe can be anything that binds to the specific target analyte. For example, the analyte binding domain can be an antibody, or antigen binding fragment thereof, capable of binding to the specific target analyte. Thus, the analyte binding domain of the first probe can be a target analyte specific antibody. In some aspects, the analyte binding domain can be, but is not limited to a protein, peptide, or nucleic acid sequence known to bind the specific target analyte.

In some aspects, the polynucleotide of the first probe comprises a sequence complementary to a portion of the polynucleotide of the second probe. In some aspects, the polynucleotide of the first probe can be bound to the analyte binding domain of the first probe. In some aspects, the polynucleotide of the first probe can be bound to the solid support of the first probe. In some aspects, the polynucleotide of the first probe can be attached on its 5′ end thus allowing extension from the 3′ end. In some aspects, the polynucleotide of the first probe can be bound to the ligand and the analyte binding domain. Examples of where the polynucleotide can be bound in the first probe can be seen in FIG. 7. In some aspects, the polynucleotide can further comprise a blocking oligonucleotide, an example of which is shown in FIGS. 4 and 7. In some aspects, the polynucleotide of the first probe comprises a barcode sequence. In some aspects, the barcode sequence is specifically associated with a target analyte. In some aspects, the barcodes can sometimes be referred to as unique molecular identifiers (or UMIs). Unique molecular identifiers (UMIs) are a method for counting molecules in single-cell RNA sequencing (scRNA-seq). UMIs can be random sequences added to DNA before amplification and sequencing to distinguish between molecules within a cell. The barcode can be used for PCR/sequencing error correction via molecular counting. Methods for molecular counting using UMIs are known in the art and can include, but are not limited to UMI method, TRUmiCount, DAUMI and AmpliCI.

ii. Second Probe

In some aspects, the second probe can be identical to the second probe used in the above methods. In some aspects, the second probe comprises an analyte binding domain specific for a target analyte and a polynucleotide.

In some aspects, the analyte binding domain of the second probe can be specific for the target analyte in the captured first complex. In some aspects, the analyte binding domain of the second probe can be anything that binds to the specific target analyte. For example, the analyte binding domain can be an antibody, or antigen binding fragment thereof, known to bind the specific target analyte. Thus, the analyte binding domain of the first probe can be a target analyte specific antibody. In some aspects, the analyte binding domain can be, but is not limited to a protein, peptide, or nucleic acid sequence known to bind the specific target analyte.

In some aspects, the analyte binding domain of the second probe can bind to a different epitope of the specific target analyte than the analyte binding domain of the first probe.

In some aspects, the polynucleotide of the second probe comprises a sequence complementary to a portion of the polynucleotide of the first probe. In some aspects, the polynucleotide of the second probe can be bound to the analyte binding domain of the second probe. In some aspects, the polynucleotide of the second probe can be attached on its 5′ end thus allowing extension from the 3′ end. In some aspects, the polynucleotide can further comprise a blocking oligonucleotide, examples of which are shown in FIGS. 4 and 7.

In some aspects, the analyte binding domain of the second probe can be the same as the analyte binding domain of the second probe of the method described above. In some aspects, the analyte binding domain of the second probe can be specific for the target analyte in the captured first complex. In some aspects, the analyte binding domain of the second probe can be anything that binds to the specific target analyte. For example, the analyte binding domain can be an antibody, or antigen binding fragment thereof, known to bind the specific target analyte. Thus, the analyte binding domain of the first probe can be a target analyte specific antibody. In some aspects, the analyte binding domain can be, but is not limited to a protein, peptide, or nucleic acid sequence known to bind the specific target analyte.

In some aspects, the analyte binding domain of the second probe can bind to a different epitope of the specific target analyte than the analyte binding domain of the first probe.

iii. Blocking Hybridization

In some aspects, the methods further comprise blocking the hybridization of the polynucleotide of the first and second probes from occurring too soon the same as described for the above method. False-positives can result if the polynucleotide of the first and second probes hybridize before the second complex is formed (for example, in the absence of target analyte). In some aspects, hybridization can be blocked using any known techniques, such as, but not limited to, using a blocking oligonucleotide, incorporating a hairpin loop into one or both polynucleotides of the first or second probe, adding a blocking reagent to the reaction mixture or adding In situ hybridization (ISH) blockers to the reaction mixture.

In some aspects, the polynucleotide of the first probe, second probe, or both can further comprise a blocking oligonucleotide. In some aspects, the blocking oligonucleotide is complementary to a portion of the polynucleotide of the first probe. In some aspects, the blocking oligonucleotide is complementary to a portion of the polynucleotide of the second probe. In some aspects, there are two blocking oligonucleotides, wherein one is complementary to a portion of the polynucleotide of the first probe and one is complementary to a portion of the polynucleotide of the second probe.

In some aspects, the blocking oligonucleotide hybridizes to a portion of the polynucleotide of the first or second probe to which it is complementary. In some aspects, the complementary portion, and thus the hybridization of the blocking oligonucleotide, is at the 3′ end of the polynucleotide of the first or second probe. Because extension can occur at the 3′ end of the polynucleotide the presence of the blocking oligonucleotide hybridized at the 3′ end of the polynucleotide of the first or second probe can prevent extension until the exact time needed.

In some aspects, the polynucleotide of the first probe, second probe, or both can further comprise a hairpin loop at the 3′ end of the polynucleotide. In some aspects, a hairpin loop is a secondary structure formed in the polynucleotide of the first probe, second probe, or both where a section of the polynucleotide folds back on itself to form base pairs with a complementary sequence within the polynucleotide. In some aspects, the polynucleotide of the first probe, second probe, or both comprises a first sequence complementary to a second sequence of the same polynucleotide, thus allowing it to hybridize with itself.

In some aspect of the methods involving the blocking oligonucleotide or hairpin loop, the methods can further comprise removing the blocking oligonucleotide or hairpin loop prior to incubating the second complex under conditions to allow for hybridization of the polynucleotide of the first probe to the polynucleotide of the second probe. In some aspects, removing the blocking oligonucleotide or hairpin loop comprises heat, enzymatic digestion, or introduction of a denaturant. For example, the method can comprise increasing the temperature of the reaction to allow denaturation of the blocking oligonucleotide or hairpin loop.

In some aspects, the blocking oligonucleotide comprises uracil residues dispersed throughout the sequence. Therefore, in some aspects, removing the blocking oligonucleotide can comprise uracil-specific endonuclease digestion. In some aspects, a similar strategy can be used for the hairpin loop.

### C. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more of the components of the claimed methods. For example, the kits can contain one or more of the first probes, second probes, capture molecules, polynucleotides, or blocking oligonucleotides described herein.

### D. Analysis Techniques to Integrate-Omics Datasets

In some aspects, the disclosed methods can further comprise -omics dataset integration

In some aspects, the disclosed methods can further comprise integrating -omics datasets from various sources, such as genomic, epigenomic, transcriptomic, and highly sensitive proteomic readouts, can provide a more comprehensive understanding of disease etiology, mechanisms of disease evolution and may better help to identify complex relationships between different molecular layers. For example, how a change in the genetic sequence can affect the epigenome or the proteome. In the discussion that follows the analysis techniques are used to integrate -omics datasets.

In some aspects, the disclosed methods can further comprise multi-omics data fusion, wherein data from different -omics layers (e.g., genomics, epigenomic, transcriptomics, and proteomics) is integrated into a unified framework. For example, such methods can be achieved through various statistical and computational techniques, such as canonical correlation analysis (CCA) or principal component analysis (PCA), to identify common patterns and relationships between the various datasets.

In some aspects, the disclosed methods can further comprise Network Analysis. For example, network analysis can be used to build biological networks (e.g., gene regulatory networks, protein-protein interaction networks) from -omics datasets and then integrating these networks can reveal how different molecular components interact with each other. Network-based methods can highlight key regulators (e.g., module eigengene which is defined as the first principal component of the expression matrix of the corresponding module) and pathways. Network analysis software can include Weighted Gene Co-expression Network Analysis (WGCNA).

In some aspects, the disclosed methods can further comprise pathway analysis to identify functional relationships between genes, transcripts, and proteins. Tools like gene set enrichment analysis (GSEA) and pathway analysis software can be used to identify significantly enriched pathways.

In some aspects, the disclosed methods can further comprise machine learning algorithms, such as random forests, support vector machines, and neural networks, can be trained on multi-omics data to classify samples, make predictions, or discover patterns. These models can integrate multiple data types which may improve accuracy.

In some aspects, the disclosed methods can further comprise preprocessing and/or normalizing omics datasets to ensure that they are on the same scale and have comparable distributions and to minimize technical biases and enhances compatibility.

In some aspects, the disclosed methods can further comprise dimensionality reduction techniques such as principal component analysis (PCA) or t-distributed stochastic neighbor embedding (t-SNE) to reduce the dimensionality of multi-omics data while preserving the most informative features.

In some aspects, the disclosed methods can further comprise Cross-Omics Correlation Analysis to identify correlations and associations between data from different -omics layers can reveal how genetic variations affect gene expression, protein levels, and other molecular phenotypes.

In some aspects, the disclosed methods can further comprise integration with clinical data. Linking -omics data to clinical information (e.g., patient outcomes, disease status, treatment response) can help identify biomarkers, predict disease progression, and personalize treatment strategies.

Similarly in temporal analysis -omics datasets collected at different time points are integrated and may provide insights into dynamic biological processes, such as changes in gene expression over time in response to a treatment, disease progression or recurrence.

In some aspects, the disclosed methods can further comprise visualizing multi-omics data through heatmaps, scatter plots, or network diagrams can aid in the exploration and interpretation of integrated results.

Moreover, analysis pipelines can be customized tailored to the specific research question and datasets may provide greater control and flexibility in -omics data integration within the disclosed methods.

Such -omics dataset integration methods can lead to a deeper understanding of the biological mechanisms underlying disease in particular cancer stage, driver mutations, recurrence, and response to therapy, tumor microenvironment, Tumor-immune interactions, angiogenesis, metastasis, micrometastases, etc. Omics integration approaches can also lead to discovery of predictive biomarkers to inform clinicians about the likelihood of a specific treatment's success in an individual patient to inform treatment selection, prognostic biomarkers to inform in overall disease course and to help prognosis and risk stratification, and diagnostic biomarkers to confirm the presence or absence of a specific disease.

In some aspects, the disclosed methods can further utilize prognostic markers in cardiovascular disease (CVD) to provide information about the expected clinical outcome or disease progression in individuals with cardiovascular diseases, help estimate the patient's prognosis, including for example the risk of future events such as heart attacks or strokes. Similarly, risk stratification biomarkers to help predict the likelihood of future cardiovascular events and guide treatment decisions.

The methods described herein can also be used to identify or detect disease risk factors like smoking status, obesity, diabetes, hypertension, and physical inactivity, which are associated with an increased risk of developing and progressing cardiovascular diseases.

### E. Computer-Readable Media

In some aspects, the disclosed methods can further comprise using computer systems (which may include one or more computers) to classify a sample from a patient is described. During operation, the computer system may receive information corresponding to at least one -omics dataset from a patient. Then, the computer system may determine a patient profile/signature based at least in part on the -omics information. Moreover, determining the patient profile/signature may include normalizing -omics datasets to ensure that they are on the same scale and have comparable distributions. Next, the computer system may use a machine learning algorithm comprising random forests, support vector machines, and/or neural networks to generate a patient profile/signature. The computer system may then compare the patient profile/signature to a reference normal signature to determine at least one classification for the sample. Note that pathway analysis to identify functional relationships between patient genes, transcripts, and/or proteins may also be used.

The present analyses are also useful in determining the efficacy of a particular treatment option. Successful treatment options may increase the amount of copy number variation or rare mutations detected in subject's blood if the treatment is successful as more cancers may die and shed DNA. In other aspects, this may not occur. In another example, perhaps certain treatment options may be correlated with genetic profiles of cancers over time. This correlation may be useful in selecting a therapy. Additionally, if a cancer is observed to be in remission after treatment, the present methods can be used to monitor residual disease or recurrence of disease.

All the methods disclosed are amenable to miniaturization, automation, and hardware integration to reduce manual processing and improve assay consistency, and reduce costs.

### EXAMPLES

### A. Example 1: Elimination of False-Positive Oligo-Mediated Proximity Extension in the Absence of Target Analyte by Means of a Tertiary Blocking Oligonucleotide that Reversibly Associates with the Antibody-Linked Oligonucleotides

Limitations of conventional immunoassays include premature hybridization-driven association of antibody reagents, resulting in false-positives. Additionally, such assays suffer from sensitivity to matrix-effects, as the proximity extension reaction typically occurs in a heterogeneous milieu, such as serum. This complex reaction matrix may inhibit or cause variability in the efficacy of enzymatic DNA-processing steps, leading to increased assay noise and variability.

A solution to the problem above is elimination of false-positive oligo-mediated proximity extension in the absence of target analyte by means of a tertiary blocking oligonucleotide that reversibly associates with the antibody-linked oligonucleotides. The association of the blocking oligonucleotide prevents direct hybridization of the two antibody-linked oligonucleotides. The blocking can be reversed by enzymatic digestion of the blocking oligonucleotide, introduction of a denaturant or by raising the temperature of the reaction. Once the deblocking reaction occurs the proximity ligation and extension events can occur. The blocking oligo digestion step can occur prior to or contemporaneously with the extension step. In a preferred embodiment the blocking oligonucleotide comprises introduction of uracil residues dispersed throughout the sequence, rendering it sensitive to uracil-specific endonuclease digestion. Blocking oligos may be used in various embodiments. FIG. 6 and FIG. 7(a)-(c) show examples of blocking oligos in proximity extension assays.

### B. Reduce Assay Variability and Noise by Temporal Separation of “Capture,” “Detection,” and Extension Steps by Means of Magnetic Separation of Target Analytes

Reducing assay variability and noise by temporal separation of “capture”, “detection” and extension steps by means of magnetic separation of target analytes. The sample (e.g. serum, plasma, blood) is initially contacted with an analyte-specific antibody, chemically linked with- or associated with a magnetic particle. The same magnetic particle is linked with an oligonucleotide, comprising a barcode sequence specifically associated with the protein analyte and capable of participating in a proximity-extension reaction, specific to the target analyte. This oligonucleotide is temporarily blocked with blocker oligonucleotide during the course of the immunoassay. The sample is then placed in a magnetic field, allowing for the selective capture of analytes bound to the antibody-associated magnetic particle. This allows removal of compounds present in the sample that could inhibit immunocomplex formation. The magnetic particle immunocomplex is contacted with a detection antibody linked to a complementary oligonucleotide, capable of participating in a proximity extension reaction. The formed immunocomplex is magnetically separated from excess detection antibody, thereby reducing risk of assay noise from free detection reagent in solution. The immunocomplex, is resuspended in a buffer amenable to enzymatic DNA-processing steps, and the blocking oligonucleotide selectively digested. The final immunocomplex brings the oligonucleotides associated with the particle surface and detection antibody into close proximity, allowing the generation of a proximity extension product mediated by a DNA polymerase enzyme. Alternatives to magnetic particles may include protein A/G agarose beads, protein A/G sepharose beads, protein L beads, streptavidin or avidin beads, protein magnetic microplates, protein capture resins, nanobodies, cellulose beads, monoclonal antibodies, antigen-purified antibodies. FIGS. 2-5 show examples embodiments using temporal separation of “capture”, “detection” and extension steps by means of magnetic separation of target analytes.

### C. High Sensitivity Multiplexed Detection and Quantitation of Cytokines and Cancer-Related Biomarkers in Blood, Serum, or Plasma

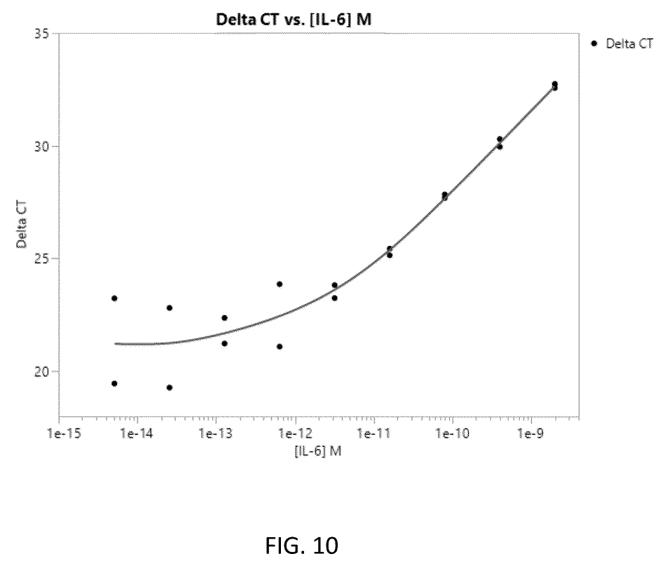

In one embodiment of the method, detection and quantification of cytokines and cancer-related biomarkers is performed in the following manner. First a 5′ amine-terminated capture oligonucleotide is reacted with an active-ester of a PEGylated biotinylation reagent and purified. Then a capture antibody is reacted with an active-ester of a PEGylated biotinylation reagent and purified. The detection antibody is chemically linked to detection oligonucleotide by means of a suitable heterofunctional cross-linking agent known to those skilled in the art. The biotinylated capture antibody and capture oligonucleotide, at a predetermined ratio and molar excess, are simultaneously mixed with a streptavidin-coated magnetic microparticle to form the capture reagent complex. The capture complex is then contacted with an excess of dideoxy-terminated uracil-rich blocker oligonucleotide. Then a plasma sample containing an analyte (e.g., cytokines or cancer related biomarker) of unknown abundance is contacted with the capture reagent for one hour, alternatively the reaction can occur for at least 30 min at room temperature or overnight at 4 C. The reaction is then followed by magnetic separation and purification using methods known in the art. The immunocomplex is then contacted with detection antibody oligonucleotide conjugate for 1 hour, alternatively, the reaction can occur for at least 30 min at room temperature or overnight at 4° C., followed by magnetic separation, purification, and suspension in a suitable buffer for molecular biology manipulations, whilst preserving the immunocomplex integrity. The immunocomplex is incubated with a solution comprising a uracil-specific digestion enzyme, an extending polymerase, nucleotides and cofactors, causing extension of the two proximity probes. Next the extension product is amplified through PCR and appended with sequences required for downstream DNA sequencing and error correction (UMIs). The amplified product is purified and sequenced by NGS on a suitable platform. The number of molecules of analyte is determined bioinformatically and related to a suitable reference or standard curve acquired contemporaneously with the assay. FIG. 10 shows qPCR signal from IL-6 titration the solid-phase immunoassay including blocking oligo protection.

### D. Improving Analyte Quantitation by Incorporation of Molecular Barcodes into the Proximity Probe Sequence to Enable Molecular Counting

Molecular barcodes, sometimes referred to as unique molecular identifiers (or UMIs) can be integrated into the oligonucleotide portion of the probe enabling PCR/sequencing error correction via molecular counting. Methods for molecular counting using UMIs are known in the art.

### E. Solid-Phase Immunoassays are Compatible with the Tertiary-Blocking Oligonucleotide Method

Solid-phase immunoassays omitting the blocking oligo/sequence has potential advantages over current commercially available solution-based methods (e.g., standard PEA) assays in specificity, multiplex potential as described previously. In a general, example the immobilized immunoassay workflows shown in FIG. 2-5 are compatible with the tertiary blocking oligonucleotide method in FIG. 6.

### F. Capture Oligo Contains a Cis-Blocking Sequence Forming a Hairpin

Capture oligo may contain a cis-blocking sequence forming a hairpin in FIG. 7 (1) immunocomplex formation that is disrupted in FIG. 7 (2) blocking oligo removal, allowing FIG. 7 (3) hybridization and extension with detection oligo. Blocking sequence removal strategies above can be applied (e.g., cleavage of uracils in blocking sequence, denaturant addition, or temperature raising). This can be beneficial as intramolecular hybridization has greater stability than intermolecular methods to increase likelihood that capture oligo will be blocked in step (1). The hairpin loop may contain specific sequence for each capture and detection antibody pair to increase hybridization and extension specificity. Additionally, the hairpin loop may contain at least one molecular identifier or UMI to tag extension events and track molecules. Examples of immunoassays using cis-blocking sequences forming a hairpin are shown in FIG. 7.

### G. Capture Oligo is Conjugated to Capture Ab Directly

In a first set of embodiments the capture oligo is conjugated to capture antibody directly with one of the following options. In a first embodiment the magnetic bead is linked to the capture oligo which in turn is linked to the capture antibody complex. A second conjugation site from the capture oligo to the magnetic bead. This conjugation may improve the proximity of capture and detection oligo for hybridization/extension step. An example of the capture oligo conjugated to the capture Ab directly as described in this embodiment is shown in FIG. 7A. In a second embodiment a universal oligo magnetic bead “capture” complex is constructed. The added sequence region on the capture oligo that is complementary to and can hybridize to a universal oligo that is conjugated to magnetic bead through its 3′ end (different than above). The added region on capture oligo can be placed 5′ or 3′ in relation to the functional sequence of the capture oligo described above. This enables manufacture of universal oligo-conjugated bead reagent (assay consistency and scalability cost benefit) and as well potentially improves capture and detection oligo proximity leading to higher assay efficiency. In a third embodiment, a universal secondary antibody bead capture complex is constructed. An antibody (Ab) is used from a single, non-human species for all capture Ab and none for the detection antibody. Conjugate the secondary antibody (to capture antibody species) to a magnetic bead. Apply secondary antibody-magnetic bead to capture oligo-antibody to from “capture complex”. This construction has the same benefits as the previous. Note both universal capture reagents can be applied before, during or after target binding reaction. FIG. 6B shows an example of the universal-secondary antibody bead capture complex construct.

### H. Target Analyte May have More than One Capture and/or Detection Antibody/Oligos, Specific Antibody Sandwich Pairing is Resolved in Readout (by Sequencing or qPCR)

Target analyte may have more than one capture and/or detection antibody/oligos, specific antibody sandwich pairing that can be resolved by NGS or qPCR. (1) Different antibodies are specific to different protein isoforms and/or post-translational modifications (PTM). An example of such antibody potential can be found in the clinical study on pancreatic cancer detection showing orthogonal signal from detection antibodies targeting different specific post translational modifications (PTMs) in CA19-9 (Partyka et al. Diverse monoclonal antibodies against the CA 19-9 antigen show variation in binding specificity with consequences for clinical interpretation. Proteomics 12, 2212-2220 (2012)), which is hereby incorporated by reference in its entirety. Such modifications/increased structural information from protein signal, can be clinically relevant (e.g., isoforms or post translational modifications) and can increase sensitivity for targets with structural variation affecting binding. Additionally, cross-reactivity of a given antibody with another protein target in the panel may be well characterized and utilized as signal to improve sensitivity. As shown in FIG. 7A if determined detection antibody 1 binds protein target 1 and target 2 reproducibly, then assay signal from the oligo extension product of detection antibody 1 and capture antibody 1, should indicate the presence of protein target 1, and extension product of detection antibody 1 and capture antibody 2 should indicate the presence of protein target 2.

### OTHER EMBODIMENTS

Embodiment 1: Disclosed are methods for preparing target analyte for sequencing, comprising: obtaining a sample comprising a plurality of target and non-target analyte; contacting the sample with at least one first probe, wherein the first probe comprises a ligand site, an analyte binding domain with affinity for at least one analyte in the sample, and a polynucleotide domain with at least one hybridization domain; allowing the first probe to bind the target analyte, thereby generating a first complex comprising analyte bound to the binding domain of the first probe; immobilizing the first complex on a solid support that comprises at least one capture molecule with affinity to bind the first complex at the ligand site, thereby linking the capture molecule to the ligand site, thereby immobilizing the first complex; removing the non-target analyte from the sample; contacting the first complex from (c) with at least a second probe, wherein the second probe comprises an analyte binding domain with affinity for the analyte in the first complex and a polynucleotide domain with at least one hybridization domain, thereby generating at least one second complex comprising analyte bound to a pair of probes; generating a polynucleotide duplex from the hybridization of the two polynucleotide domains; amplifying the polynucleotide duplex to generate a population of polynucleotide tags; and sequencing the polynucleotide tags to generate sequencing reads.

Embodiment 2: The method of Embodiment 1, wherein the analyte is a protein, a polypeptide, or a macromolecular complex comprising at least two proteins.

Embodiment 3: The method of Embodiment 1, wherein the analyte comprises a proteomic signature of cancer.

Embodiment 4: The method of Embodiment 1, wherein the analyte comprises a proteomic signature of early-stage cancer.

Embodiment 5: The method of Embodiment 1, wherein the sample comprises a mixture of biomolecules.

Embodiment 6: The method of Embodiment 1, wherein the sample comprises plasma.

Embodiment 7: The method of Embodiment 1, wherein the first probe comprises an antibody.

Embodiment 8: The method of Embodiment 7, wherein the antibody comprises a monoclonal or a polyclonal antibody.

Embodiment 9: The method of Embodiment 7 or 8, wherein the antibody comprises a recombinant antibody.

Embodiment 10: The method of Embodiment 1, wherein the ligand comprises biotin, polyhistidine tag, glutathione-S-transferase (GST), and/or a synthetic tag.

Embodiment 11: The method of Embodiment 1, wherein the analyte binding domains of the first and second probes have innate affinity for the analyte or are engineered to have affinity for the analyte in the sample.

Embodiment 12: The method of Embodiment 11, wherein the engineered affinity comprises introducing a change in one or more amino acids of the analyte binding domain.

Embodiment 13: The method of Embodiment 1, wherein the polynucleotide domains of the first and second probes comprise at least one barcode.

Embodiment 14: The method of Embodiment 1, wherein the hybridization domains of the first and second probes comprise at least one barcode.

Embodiment 15: The method of Embodiment 1, wherein the solid support comprises a magnetic particle or a magnetic bead.

Embodiment 16: The method of Embodiment 1, wherein the capture molecules in the solid support comprise streptavidin.

Embodiment 17: The method of any one of the preceding Embodiments, wherein the sample comprises one or more biological samples extracted from a sample from a patient.

Embodiment 18: The method of Embodiment 17, wherein the patient is a cancer patient.

Embodiment 19: The method Embodiment 17 or 18, wherein the cancer is selected from the group consisting of biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemiaF (ALL), acute myeloid leukemia (AML), chronic lymphocytic (CLL), chronic myeloid (CML), chronic myelomonocytic (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, and uterine sarcoma.

Embodiment 20: The method of any one of Embodiment 17-18, wherein the sample is selected from the group consisting of blood, serum, plasma, bone marrow aspirate, bile, cerebral spinal fluid (CSF), saliva, urine.

Embodiment 21: A method for preparing an analyte for sequencing, comprising: linking at least one set of first and second probes to a respective site on the analyte, wherein the probes comprise an analyte binding domain with affinity for the one or more analyte in the sample, wherein the first probe further comprises a nucleic acid domain, wherein the second probe further comprises a solid support coupled to at least one nucleic acid domain that is hybridized to a blocking oligo thereby preventing the nucleic acid domains in the set of probes to hybridize; removing the blocking oligo enabling the nucleic acid domains in the set of probes to hybridize and form a nucleic acid duplex; amplifying the nucleic acid duplex to generate a population of nucleic acid tags; and sequencing nucleic acid tags to generate sequencing reads.

Embodiment 22: The method of Embodiment 21, wherein the blocking oligo further comprises uracil residues dispersed throughout the sequence.

Embodiment 23: The method of Embodiment 21, wherein the removing further comprises uracil-specific endonuclease digestion.

Embodiment 24: The method of Embodiment 21, wherein the removing comprises enzymatic digestion of the blocking oligonucleotide, introduction of a denaturant, or increasing the temperature of the reaction to allow denaturation of the blocking oligo.

Embodiment 25: The method of any one of the preceding Embodiments, wherein the probe comprises at least one aptamer.

Embodiment 26: The method of any one of the preceding Embodiments, wherein at least one portion of the probe comprises at least one aptamer.

Embodiment 27: The method of Embodiment 4, wherein the proteomic signature of early-stage cancer is integrated with multi-omics datasets and/or patient metadata using random forests, support vector machines, or neural networks to generate at least one classification for the sample.

Embodiment 28: The method of Embodiment 27, wherein the multi-omics datasets are selected from the group consisting of: RNA-Seq, ChIP-Seq, CUT&Tag sequencing, Hi-C-Seq, DNA-Seq, Whole Exome Sequencing (WES), Whole Genome Sequencing (WGS), Metagenomics Sequencing, Bisulfite Sequencing, Small RNA Sequencing, Single-Cell RNA Sequencing, RAD-Seq, ATAC-Seq, Cap-Seq, MeDIP-Seq, RIP-Seq, methylation data, fragmentomic data, metabolomic data, or lipidomic data.

Embodiment 29: The method of Embodiment 27, wherein the metadata is selected from the group consisting of: age, BMI, ethnicity, smoking status, alcohol consumption status, clinical data.

Embodiment 30: The method of Embodiment 27, wherein the classification comprises a cancer status and/or a type of cancer.

Embodiment 31: The method of Embodiment 27, wherein the classification is selected from the group consisting of: stroke risk, cardiovascular disease risk, neurological disease risk.

Embodiment 32: A method, comprising: contacting a sample comprising target analyte and non-target analyte with at least one first probe, wherein the first probe comprises a ligand site, an analyte binding domain with affinity for at least one analyte in the sample, and a polynucleotide domain with at least one hybridization domain; allowing the first probe to bind the target analyte, thereby generating a first complex comprising analyte bound to the binding domain of the first probe; immobilizing the first complex on a solid support that comprises at least one capture molecule with affinity to bind the first complex at the ligand site, thereby linking the capture molecule to the ligand site, thereby immobilizing the first complex; removing the non-target analyte from the sample; contacting the first complex from (b) with at least a second probe, wherein the second probe comprises an analyte binding domain with affinity for the analyte in the first complex and a polynucleotide domain with at least one hybridization domain, thereby generating at least one second complex comprising analyte bound to a pair of probes; generating a polynucleotide duplex from the hybridization of the two polynucleotide domains; and amplifying the polynucleotide duplex to generate a population of polynucleotide tags.

Those skilled in the art will recognize, or be able to ascertain using no more than routine experimentation, many equivalents to the specific embodiments of the method and compositions described herein. Such equivalents are intended to be encompassed by the following claims.

1.-32. (canceled)

33. A method, comprising:
contacting a sample comprising one or more target analytes with at least one first probe, wherein the first probe comprises a ligand, an analyte binding domain and a polynucleotide, wherein the first probe specifically binds to one of the target analytes in the sample, thereby generating a first complex comprising the target analyte bound to the analyte binding domain of the first probe;
contacting the first complex with a capture molecule, wherein the capture molecule binds to the ligand of the first probe of the first complex, thereby generating a captured first complex;
contacting the captured first complex with at least one second probe, wherein the second probe comprises an analyte binding domain specific for the target analyte in the captured first complex and a polynucleotide, wherein the polynucleotide of the second probe comprises a sequence complementary to a portion of the polynucleotide of the first probe, wherein the second probe specifically binds to the target analyte in the captured first complex, thereby generating at least one second complex;
incubating the second complex under conditions to allow for hybridization of the polynucleotide of the first probe to the polynucleotide of the second probe; and
extending the hybridized polynucleotides of the first and second probes to generate complementary strands of the polynucleotides of the first and second probes.

34. The method of claim 33, further comprising amplifying the extended polynucleotides of step (e).

35. The method of claim 34, wherein the method further comprises immobilizing the first complex between step (b) and (c).

36. The method of claim 35, wherein the capture molecule is bound to a solid support, thereby immobilizing the captured first complex.

37. The method of claim 36, wherein the sample further comprises one or more non-target analytes.

38. The method of claim 37, further comprising removing the one or more non-target analytes from the sample.

39. The method of claim 38, further comprising removing any unbound second probes after step (c).

40. The method of claim 33, wherein the 3′ end of the polynucleotide of the second probe hybridizes to the 3′ end of the polynucleotide of the first probe.

41. The method of claim 40, wherein the polynucleotide of the first probe of the polynucleotide comprises a sequence complementary to a portion of the polynucleotide of the second probe.

42. The method of claim 40, wherein the polynucleotide of the first probe is bound to the ligand of the first probe.

43. The method of claim 40, wherein the polynucleotide of the first probe is bound to the analyte binding domain of the first probe.

44. The method of claim 63, wherein the analyte binding domain of the first probe is a target analyte specific antibody.

45. The method of claim 44, wherein the polynucleotide of the first or second probe, further comprises a blocking oligonucleotide.

46. The method of claim 45, wherein the blocking oligonucleotide is complementary to a portion of the polynucleotide of the first or second probe.

47. The method of 46, wherein the blocking oligonucleotide hybridizes to a portion of the polynucleotide of the first or second probe.

48. The method of claim 47, further comprising removing the blocking oligonucleotide prior to incubating the second complex under conditions to allow for hybridization of the polynucleotide of the first probe to the polynucleotide of the second probe, wherein removing comprises heat or enzymatic digestion.

49. (canceled)

50. The method of claim 33, wherein the polynucleotide of the first or second probe comprises a barcode sequence.

51. The method of claim 50, wherein the barcode sequence is specifically associated with a target analyte.

52. The method of claim 51, wherein the polynucleotide of the first or second probe comprises a hairpin at the 3′ end.

53. The method of claim 52, further comprising denaturing the hairpin oligonucleotide prior to incubating the second complex under conditions to allow for hybridization of the polynucleotide of the first probe to the polynucleotide of the second probe, wherein denaturing comprises heat or enzymatic digestion.

54.-94. (canceled)
